(19) United States
(12) Patent Application Publication
(10) Pub. No.: US 2010/0068821 A1
(43) Pub. Date: Mar. 18, 2010

(54) METHOD FOR DETECTION AND ANALYSIS OF AROMATIC HYDROCARBONS FROM WATER

(76) Inventor: Randy St. Germain, Fargo, ND (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/540,417

(22) Filed: Aug. 13, 2009

Related U.S. Application Data
(60) Provisional application No. 61/096,508, filed on Sep. 12, 2008.

(51) Int. Cl. G01N 33/00

(52) U.S. Cl. 436/140

(57) ABSTRACT

Methods for analyzing aromatic hydrocarbons dissolved in water are discussed. The methods include providing a substrate coated with a thin film layer of a material, wherein the material has a high affinity for at least one aromatic hydrocarbon, the material is substantially optically transparent, and the material has near-zero auto fluorescence, inserting the coated substrate directly into an environmental location including water, waiting for an exposure time permitting at least one aromatic hydrocarbon to absorb into the thin film layer, retrieving the coated substrate from the environmental location, removing any non-absorbed matter from the coated substrate, and performing fluorescence analysis on the coated substrate to detect aromatic hydrocarbons present in the thin film layer. Also methods for analyzing aromatic hydrocarbons dissolved in water contained in coated vessels are provided.

### CROSS REFERENCE TO RELATED APPLICATIONS

This application claims priority to prior U.S. Provisional Application No. 61/096,508, filed Sep. 12, 2008, which is incorporated herein by reference.

### FIELD OF THE INVENTION

The present invention relates generally to facilitating testing for environmental contaminants or other analytes by extracting from an environmental location, or from a fluid sample matrix, analytes present in trace amounts using a thin film layer of material for which the analytes of interest have a high affinity and that is substantially optically transparent and has a near-zero autofluorescence, then subjecting the thin film material with captured analytes to laser induced fluorescence (LIF) or other testing.

### BACKGROUND OF THE INVENTION

Detection and/or measurement of aromatic hydrocarbons including polycyclic aromatic hydrocarbons (PAHs) and monocyclic aromatic hydrocarbons (MAHs), considered contaminants in the environment and other analytes of interest in a fluid sample matrix, can be difficult. Many aromatic hydrocarbons are in forms that are not easily detected, or are dispersed in matrices or media that are unfit for field-work analysis, particularly for laser induced fluorescence (LIF) analysis.

For example, PAHs in coal tar and creosote are difficult to detect spectroscopically in their customary soil or water environment, because they may not fluoresce well in such media. PAHs present in murky water, sediments or soils are unsuitable for LIF or similar optical analysis.

Several investigators have demonstrated that using sediment concentrations and conventional organic carbon/water partitioning coefficients (K_{OC}) can over predict pore water concentrations of hydrophobic organic pollutants such as polycyclic aromatic hydrocarbons (PAHs) by up to three orders-of-magnitude, most likely because of the presence of several types of “black” or “soot” carbon (BC) in sediments that tightly bind PAHs. (Jonker, M. T. O.; Koelmans, A. A. Sorption of polycyclic aromatic hydrocarbons and polychlorinated biphenyls to soot and soot-like materials in the aqueous environment: Mechanistic considerations. Environ. Sci. Technol. 2002, 36, 3725-3734. Cornelissen, G.; Gustafsson, O.; Bucheli, T. D.; Jonker, M. T. O.; Koelmans, A. A.; van Noort, P. C. M. Extensive sorption of organic compounds to black carbon, coal, and kerogen in sediments and soils: mechanisms and consequences for distribution, bioaccumulation, and biodegradation. Environ. Sci. Technol. 2005, 39, 6881-6895. Khalil, M. F.; Ghosh, U.; Kreitinger, J. P. Role of weathered coal tar pitch in the partitioning of polycyclic aromatic hydrocarbons in manufactured gas plant site sediments. Environ. Sci. Technol. 2006, 40, 5681-5687. Hawthorne, S. B.; Grabanski, C. B.; Miller, D. J. Measured partitioning coefficients for parent and alkyl polycyclic aromatic hydrocarbons in 114 historically contaminated sediments: Part 1. K_{OC} values. Environ. Toxicol. Chem. 2006, 25, 2901-2911. Lohmann, R.; MacFarlane, J. K.; Gschwend, P. M. Importance of black carbon to sorption of native PAHs, PCBs, and PCDDs in Boston and New York harbor sediments. Environ. Sci. Technol. 2005, 39, 141-148.)

Therefore, investigations into the bioavailability of PAHs and related hydrophobic organics in sediments have increasingly focused on measuring pore water concentrations, rather than attempting to predict pore water concentrations based on sediment concentrations. (Xu, Y.; Spurlock, F.; Wang, Z.; Gan, J. Comparison of five methods for measuring sediment toxicity of hydrophobic contaminants. Environ. Sci. Technol. 2007, 41, 8394-8399. Hawthorne, S. B.; Azzolina, N. A.; Neuhauser, E. F.; Kreitinger, J. P. Predicting bioavailability of sediment polycyclic aromatic hydrocarbons to Hyalella azteca using equilibrium partitioning, supercritical fluid extraction, and pore water concentrations. Environ. Sci. Technol. 2007, 41, 6297-6304. Cornelissen, G.; Pettersen, Ar.; Broman, D.; Mayer, P.; Breedveld, G. D. Field testing of equilibrium passive samplers to determine freely dissolved native polycyclic aromatic hydrocarbon concentrations. Environ. Toxicol. Chem. 2008, 27(3) 499-508. Hunter, W.; Xu, Y.; Spurlock, F.; Gan, J. Using disposable polydimethylsiloxane fibers to assess the bioavailability of permethrin in sediment. Environ. Toxicol. Chem. 2008, 27(3), 568-575. Jonker, M. T. O.; Van Der Heijden, S. A.; Kreitinger, S., Hawthorne, S. B. Predicting PAH bioaccumulation and toxicity in earthworms exposed to manufactured gas plant soils with solid-phase microextraction. Environ. Sci. Technol. 2007, 41, 7472-7478. Styrishave, B.; Mortensen, M.; Krogh, P. H.; Andersen, O.; Jensen, J. Solid-phase microextraction (SPME) as a tool to predict the bioavailability and toxicity of pyrene to the springtail, Folsomia candida, under various soil conditions. Environ. Sci. Technol. 2008, 42, 1332-1336.)

Pore water concentrations are usually measured either by direct exposure of a non-depletive sorbent into the sediment/water slurry (Cornelissen, G.; Pettersen, Ar.; Broman, D.; Mayer, P.; Breedveld, G. D. Field testing of equilibrium passive samplers to determine freely dissolved native polycyclic aromatic hydrocarbon concentrations. Environ. Toxicol. Chem. 2008, 27(3) 499-508. Hunter, W.; Xu, Y.; Spurlock, F.; Gan, J. Using disposable polydimethylsiloxane fibers to assess the bioavailability of permethrin in sediment. Environ. Toxicol. Chem. 2008, 27(3), 568-575. Jonker, M. T. O.; Van Der Heijden, S. A.; Kreitinger, S., Hawthorne, S. B. Predicting PAH bioaccumulation and toxicity in earthworms exposed to manufactured gas plant soils with solid-phase microextraction. Environ. Sci. Technol. 2007, 41, 7472-7478. Styrishave, B.; Mortensen, M.; Krogh, P. H.; Andersen, O.; Jensen, J. Solid-phase microextraction (SPME) as a tool to predict the bioavailability and toxicity of pyrene to the springtail, Folsomia candida, under various soil conditions. Environ. Sci. Technol. 2008, 42, 1332-1336), or by separating the pore water and determining the dissolved PAH concentrations after solvent extraction or by using solid-phase microextraction (SPME) (Xu, Y.; Spurlock, F.; Gan, J. Using disposable polydimethylsiloxane fibers to assess the bioavailability of permethrin in sediment. Environ. Toxicol. Chem. 2008, 27(3), 568-575. Hawthorne, S. B.; Grabanski, C. B.; Miller, D. J.; Kreitinger, J. P. Solid-phase microextraction measurement of parent and alkyl polycyclic aromatic hydrocarbons in milliliter sediment pore water samples and determination of K_{DOC} values. Environ. Sci. Technol. 2005, 39, 2795-2803.)

In addition to the increasing recognition that direct pore water measurements are needed to predict the bioavailability of sediment PAHs, it is becoming apparent that the conventional parent PAHs measured by EPA method 8270 (PAH-16) are not sufficient to represent potential PAH biological effects. (Hawthorne, S. B.; Azzolina, N. A.; Neuhauser, E. F.; Kreitinger, J. P. Predicting bioavailability of sediment polycyclic aromatic hydrocarbons to Hyalella azteca using equilibrium partitioning, supercritical fluid extraction, and pore water concentrations. Environ. Sci. Technol. 2007, 41, 6297-6304. U.S. Environmental Protection Agency. Procedures for the derivation of ESBs for the protection of benthic organisms: PAH mixtures; EPA/600/R-02/013; Office of Research and Development: Washington, D.C., 2003.)

For example, the PAH-16 only account for about 40% of the total PAH concentrations in coal tars from manufactured gas plant (MGP) sources, and only about 1% of the total PAH concentrations in a petroleum crude oil. (Hawthorne, S. B.; Miller, D. J.; Kreitinger, J. P. Measurement of ‘total’ PAH concentrations and toxic units used for estimating risk to benthic invertebrates at manufactured gas plant sites. Environ. Toxicol. Chem. 2006, 25, 287-296.)

In recognition of this fact, the U.S. EPA has proposed measuring a more inclusive range of 18 parent and 16 groups of alkyl PAHs (PAH-34) in sediments and sediment pore water. (U.S. Environmental Protection Agency. Procedures for the derivation of ESBs for the protection of benthic organisms: PAH mixtures; EPA/600/R-02/013; Office of Research and Development: Washington, D.C., 2003.)

Although laboratory methods to measure pore water PAH-34 concentrations have been developed (Hawthorne, S. B.; Grabanski, C. B.; Miller, D. J.; Kreitinger, J. P. Solid-phase microextraction measurement of parent and alkyl polycyclic aromatic hydrocarbons in milliliter sediment pore water samples and determination of K_{DOC} values. Environ. Sci. Technol. 2005, 39, 2795-2803), there is a strong desire on the part of site managers and regulatory personnel to determine pore water PAH concentrations on site with in situ samplers, both to reduce the time and cost of site surveys and to minimize alterations to the samples that may occur during sample collection, shipping, and laboratory analysis.

Several groups have used a non-depletive in situ solid-phase microextraction (SPME) approach to determine dissolved PAH pore water concentrations. Sorbents such as polydimethylsiloxane (PDMS) or polyoxymethylene (POM) are inserted directly into sediment/water slurries and typically left for weeks to come to equilibrium. (Cornelissen, G.; Pettersen, Ar.; Broman, D.; Mayer, P.; Breedveld, G. D. Field testing of equilibrium passive samplers to determine freely dissolved native polycyclic aromatic hydrocarbon concentrations. Environ. Toxicol. Chem. 2008, 27(3) 499-508. Hunter, W.; Xu, Y.; Spurlock, F.; Gan, J. Using disposable polydimethylsiloxane fibers to assess the bioavailability of permethrin in sediment. Environ. Toxicol. Chem. 2008, 27(3), 568-575. Jonker, M. T. O.; Van Der Heijden, S. A.; Kreitinger, S., Hawthorne, S. B. Predicting PAH bioaccumulation and toxicity in earthworms exposed to manufactured gas plant soils with solid-phase microextraction. Environ. Sci. Technol. 2007, 41, 7472-7478. Styrishave, B.; Mortensen, M.; Krogh, P. H.; Andersen, O.; Jensen, J. Solid-phase microextraction (SPME) as a tool to predict the bioavailability and toxicity of pyrene to the springtail, Folsomia candida, under various soil conditions. Environ. Sci. Technol. 2008, 42, 1332-1336.)

The partitioning of PAHs to such sorbents is controlled primarily by each PAH's octanol/water partitioning coefficient (K_{OW}), and is therefore thought to mimic partitioning of PAHs between sediment pore water and biological lipids. Such sorbents are typically retrieved from the sediment, returned to the laboratory, and solvent extracted to determine PAH concentrations by conventional chromatographic methods. Therefore, these methods tend to retain many of the time and cost disadvantages of collecting sediment samples and shipping them to the laboratory for pore water analysis.

There have also been several attempts to directly measure PAH concentrations in water using laser-induced fluorescence (LIF). Unfortunately, the success of LIF to determine PAH concentrations has been limited by background spectral interferences from natural dissolved organic matter (DOM). (Kuo, D. T. F.; Adams, R. G.; Rudnick, S. M.; Chen, R. F.; Gschwend, P. M. Investigating desorption of native pyrene from sediment on minute- to month-timescales by time-gated fluorescence spectroscopy. Environ. Sci. Technol. 2007, 41(22), 7752-7758. Nahorniak, M. L.; Booksh, K. S. Excitation-emission matrix fluorescence spectroscopy in conjunction with multiway analysis for PAH detection in complex matrices. Analyst, 2006, 131, 1308-1315. Valero-Navarro, A.; Fernández-Sánchez, Medina-Castillo, A. L.; Fernández-Ibáñez, F.; Segura-Carretero, A.; Ibáñez, J. M.; Fernández-Gutiérrez. A rapid, sensitive screening test for polycyclic aromatic hydrocarbons applied to Antarctic water. Chemosphere 2007, 67, 903-910. Rudnik, S. M.; Chen, R. F. Laser-induced fluorescence of pyrene and other polycyclic aromatic hydrocarbons (PAH) in seawater. Talanta 1998, 47, 907-919. Kotzick, R.; Niessner, R. Application of time-resolved, laser-induced and fiber-optically guided fluorescence for monitoring of a PAH-contaminated remediation site. Fresenius J. Anal. Chem. 1996, 354, 72-76.)

Time-resolved fluorescence has been used to reduce background DOM emission, but approaches typically only measure a limited number of parent PAHs. (Kuo et al., Nahorniak et al., Valero-Navarro et al., Rudnik et al., Kotzick et al.), cited above.

An alternate approach would be to separate the PAHs from the DOM prior to LIF with the use of a non-polar solvent such as hexane (Owen, C. J.; Axler, R. P.; Nordman, D. R.; Schubauer-Berigan, M.; Lodge, K. B.; Shubauer-Berigan, J. P. Screening for PAHs by fluorescence spectroscopy: a comparison of calibrations. Chemosphere 1995, 31, 3345-3356), but this requires separation of the sediment and pore water, and is not practical in situ (embedded directly into the sediment) in the field. It also generates organic waste which is not “green”.

Another process for extracting and measuring PAHs from soil, oil, and/or water samples involves thermal desorption, which uses heat to remove PAHs from a sample matrix for subsequent analysis. However, thermal desorption processes destroy the environmental sample, and a single test of a selected sample may be performed (unless the sample is large and PAHs are uniformly distributed in it). In addition thermal desorption is certainly not applicable in situ.

It is sometimes desirable to detect PAHs or other analytes in fluid flows where monitoring the changes of PAHs or other analytes in the fluid over time or space are of interest. In such environments, one known process for measuring PAHs in a fluid involves optically monitoring PAHs in water flowing through a pipe or other conduit equipped with a window. However, optical monitoring technologies are hindered because the fluid is often cloudy or even opaque, reducing the volume being optically integrated. Additionally, optical windows may become contaminated, making optical measurement and/or detection of PAHs present in the flow difficult.

Yet another process for extracting and measuring analytes from the environment includes solid-phase micro extraction (SPME) of analytes, followed by analysis of the SPME material capturing the analytes extracted from the sample matrix. In one example, analytes may be extracted and analyzed using spectral analysis by extracting analytes from aqueous samples using stir-bar sorptive extraction, in which a stir bar is coated with a layer of absorbent material capable of absorbing organic compounds present at low, trace levels in aqueous matrices (e.g., polydimethylsiloxane or PDMS) and subsequently analyzed to determine the presence of absorbed organic compounds. (David et al., Stir-Bar Sorptive Extraction of Trace Organic Compounds from Aqueous Matrices, LCGC North America, February 2003).

However, stir-bar sorptive extraction requires removal of aqueous samples from an environment to a testing lab so that the stir-bar may be actuated in the aqueous sample. After exposure to the sample, the stir-bar is subjected to thermal desorption to deliver thermally desorbed analytes to a gas chromatograph. In addition, with stir-bar sorptive extraction, accurate determination of analytes at different levels of a core sampling is expensive and time consuming.

Analytes in aqueous media also may be extracted via absorption into a solid block or sheet of solid phase extraction (SPE) material and subsequently detected using synchronous fluorescence analysis directly on the solid phase (or a slice thereof) after extraction. (See Algarra et al., Direct Fluorometric Analysis of PAHs in Water an in Urine Following Liquid Solid Extraction, J. Fluorescence 355-359, 2000.) However, blocks of PDMS used are relatively thick and not well suited for quick spectral analysis or small sample analysis, because of long exposure times and depletion of small samples (or the zone immediately surrounding the sampler) and thick blocks of SPE material need to be processed and prepared for spectral analysis. Neither are sheets suitable for in situ field testing, because of damaging the very thin sheets required while inserting into, retrieving from, and removing sediments and soils from the sampler.

U.S. Pat. No. 7,222,546, issued to St. Germain, incorporated herein by reference, describes a method and apparatus for sediment characterization in which an elongated sampler made of or coated with PDMS is positioned in sediment. Where PAHs or similar analytes in NAPLs, solids or aqueous phase touch the sampler, they are absorbed into the sampler. In effect, this creates a contact print of the distribution of PAHs along the sampler's length. The sampler can be removed from the sediment and the captured analyte's contact print “read” at any location along its length. LIF is one useful reading method. However, such samplers cannot easily be monitored in situ during sorption to observe absorption rates. Moreover, the physically robust forms of PDMS rubber that are required for use (in order to prevent tearing, breakage, or other destruction during insertion/retrieval) contain amendments or additives that produce significant autofluorescence. Due to this relatively high background fluorescence, these elongated samplers only reliably detect analytes at concentrations attained when the device is in contact with PAH NAPLs (NAPLs hold thousands to millions of times higher concentrations of PAHs due to PAH's having solubility in oils rather than in water) or high dissolved phase PAHs (orders of magnitude above toxic levels). While aqueous phase PAHs are absorbed, their limit of detection is hindered because their fluorescence emission is overwhelmed by the high background fluorescence of strong/durable forms of PDMS.

Because the PAH-containing NAPL phase of coal tars, creosotes, and crude oils can contain hold thousands to millions of times more PAH than water can, the NAPL is often referred to as the “source term” meaning the source of the dissolved phase PAHs that can continue to supply PAHs to the pore water for decades, even centuries. It is important to investigators and environmental risk analyzers to determine if there is even tiny quantities of NAPL in the environment or sample. One commonly used technique is to add an oil soluble hydrophobic dye such as Sudan IV to a soil or sediment sample, shake/mix the sample, then examine for presence of the orange-red color, indicating presence/absence of NAPL. However, this test is subjective, and is often difficult on optically dense or dark contaminants like coal tars and creosotes because their inherent optical density limits the volume of NAPL the human eye can interrogate for the red-orange color of the dissolved dye. Relative to the dye test, PAH fluorescence that results from PDMS exposed to PAH-containing NAPL is a much more intense phenomenon readily observed with fluorescence instrumentation or “machine vision” which allows for lower detection limits and improved reproducibility.

Therefore, a need exists for a SPE technique with substrate configurations that may be flexibly used in a range of testing situations and for methods of detecting and/or measuring contaminants and other analytes directly in environmental locations and in sample matrices taken from environmental locations using SPE substrate configurations that are simple, inexpensive, and fast.

### BRIEF SUMMARY OF THE INVENTION

The present invention provides a simple, field-portable method to determine total dissolved aromatic hydrocarbon concentrations in water, including turbid fluids, surface waters, groundwater and even sediment pore water. It is based on in situ sampling with solid-phase microextraction (SPME) coupled with analysis by fluorescence.

Advantageously, the field-portable method determines pore water aromatic hydrocarbon concentrations independent of the presence of polar dissolved organic matter or of sample size to allow fluorescent analysis of sediment pore water PAH-34 concentrations while still at the environmental location.

Additionally, the present invention also provides for a system and method for extraction of trace amounts of contaminants or other analytes found in fluid sample matrices for subsequent analysis, such as detection and measurement by fluorescence analysis, or in particular laser induced fluorescence (LIF).

While a variety of analytes in a variety of sample matrices may be tested using the methods and systems described, testing for aromatic hydrocarbons in situ in sample matrices, sample matrices taken from the environment or from flows in the environment or in various industrial processes are areas of application.

According to certain embodiments, methods are provided for analyzing aromatic hydrocarbons dissolved in water including providing a substrate coated with a thin film layer of a material, wherein the material has a high affinity for at least one aromatic hydrocarbon, the material is substantially optically transparent, and the material has near-zero auto fluorescence, inserting the coated substrate directly into an environmental location including water, waiting for an exposure time permitting at least one aromatic hydrocarbon to absorb into the thin film layer, retrieving the coated substrate from the environmental location, removing any non-absorbed matter from the coated substrate, and performing fluorescence analysis on the coated substrate to detect aromatic hydrocarbons present in the thin film layer.

In one aspect, the material of the thin film layer is polydimethylsiloxane (PDMS).

In another aspect, the PDMS has a thickness of about 50 μm.

In yet another aspect, the fluorescence analysis is laser induced fluorescence (LIF) analysis.

In one embodiment the LIF analysis has a detection limit for aromatic hydrocarbons of about 0.2 ng/mL.

In some embodiments the aromatic hydrocarbons are polycyclic aromatic hydrocarbons.

In another embodiment the polycyclic aromatic hydrocarbons are bioavailable polycyclic aromatic hydrocarbons.

In some embodiments, the fluorescence analysis on the coated substrate detects 2-ring and 3-ring polycyclic aromatic hydrocarbons present in the thin film layer.

In one aspect, the aromatic hydrocarbons are 2-ring to 6-ring polycyclic aromatic hydrocarbons.

In another aspect, the aromatic hydrocarbons are monocyclic aromatic hydrocarbons.

In yet another aspect, the concentrations of aromatic hydrocarbons absorbing into the coating layer are relatively independent of water sample size.

In one embodiment the concentrations of aromatic hydrocarbons absorbing into the thin film layer occur independently of the presence of non-aqueous phase liquids.

In another embodiment the concentrations of aromatic hydrocarbons absorbing into the thin film layer are independent of the presence of dissolved organic matter.

In some embodiments the substrate is an optical fiber.

In one embodiment the substrate is magnetic.

In some embodiments methods are provided for analyzing aromatic hydrocarbons dissolved in water including providing a substantially optically transparent substrate coated with a thin film layer of a material, wherein the material has a high affinity for at least one aromatic hydrocarbon, the material is substantially optically transparent, and the material has near-zero auto fluorescence, inserting the coated substrate into an environmental location including water, waiting for an exposure time permitting at least one aromatic hydrocarbon to absorb into the thin film layer to equilibrium, retrieving the coated substrate from the environmental location, removing any non-absorbed matter from the coated substrate, and performing analysis on the coated fiber with a fluorometer to detect aromatic hydrocarbons present in the thin film layer.

In one embodiment methods are provided for analyzing aromatic hydrocarbons present in a fluid sample matrix, including, providing a vessel, providing a thin film layer in the vessel, wherein the thin film layer comprises polydimethylsiloxane that at least one aromatic hydrocarbon has a high affinity for and that is substantially optically transparent and has near-zero autofluorescence in the vessel, collecting a fluid sample matrix from an environmental location, placing the fluid sample matrix in the vessel, periodically exposing at least one sampling point on the thin film layer to an excitation light and sensing a corresponding laser induced fluorescence (LIF) response, storing a time sequence of LIF responses from the at least one sampling point, and determining from the time sequence of LIF responses when an equilibrium has been sufficiently achieved for absorption of the at least one aromatic hydrocarbon into the thin film layer.

In one aspect, the thin film layer is coated on an inner surface of the vessel.

In another aspect, a plurality of coated vessels are provided on a carousel for collecting sample matrices over time.

In some embodiments the vessel is at least one of a jar, a bag or a tube.

In one embodiment the vessel is made from a substantially optically transparent material and the LIF exposing and sensing occurs with light passing through the vessel.

In another embodiment the vessel has a substantially optically transparent portion, and the thin film layer is coated on the portion.

In some embodiments methods are provided for analyzing aromatic hydrocarbons dissolved in water including providing a substrate coated with a thin film layer of a material, wherein the material has a high affinity for at least one aromatic hydrocarbon, the material is substantially optically transparent, and the material has near-zero auto fluorescence, forming the coated substrate as a recording medium stored on a spool or in a cassette, transporting the recording medium having a plurality of coated segments in and out of an environmental location including water, exposing one or more of the plurality of coated segments to absorption of aromatic hydrocarbons from the water into the one or more coated segments, successively drawing the plurality of coated segments into an analyzer, and analyzing an exposed coated segment of recording medium with a fluorometer to detect aromatic hydrocarbons present in the thin film layer.

In some aspects, the recording medium is in the form of a tape, wire or string.

In another aspect, the material of the thin film layer comprises polydimethylsiloxane.

These and other features and advantages of the present invention will become apparent to those skilled in the art from the following detailed description, wherein it is shown and described illustrative embodiments of the invention, including best modes contemplated for carrying out the invention. As it will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

The invention will be more fully understood from the following detailed description taken in conjunction with the accompanying drawings, in which:

FIG. 1a is an illustration of a SPME coated rod or needle inserted into sediment at the bottom of a jar and shows a field deployment sampler inserted at the bottom of a body of water.

FIG. 1b is an illustration of how FIG. 1a appears under fluorescence analysis after the SPME coated rod or needle absorbs fluorescing analytes.

FIG. 1c is an illustration of a front and side view of an SPME coated strip in a development vessel holding a sample matrix to be tested, and shows front and side views of an SPME strip in a development vessel holding a sample matrix.

FIG. 1d is an illustration of how FIG. 1c appears under fluorescence analysis after the SPME strip absorbs fluorescing analytes.

FIG. 2 is an illustration of an SPME coated wire/string fed from a spool into a flow environment.

FIG. 3 is an illustration of a the kind of graph showing a log of PAH content vs. time that may be derived from the device of FIG. 2.

FIG. 4a provides a flowchart of a method for analyzing trace contaminants present in a sample matrix.

FIG. 4b provides another flowchart of a method for analyzing trace contaminants present in a sample matrix.

FIG. 4c provides a flowchart of a method for detecting the presence of contaminants according to certain implementations.

FIG. 4d provides a flowchart of a method for analyzing trace contaminants present in a fluid sample matrix in situ.

FIG. 5 provides a graph of results from one SPME device in one sample.

FIG. 6 provides a graph of results for SPME devices from a plurality of vessels in a carousel.

FIG. 7 is an illustration of a number of development vessels, e.g., 17 development vessels, each having been coated internally with a PDMS thin film sampler layer are provided on a carousel.

FIG. 8 provides a graph of results from testing with a hand held fluorometer compared to testing with laser induced fluorescence.

FIG. 9a is an illustration of bench top use of a magnetic retrieval device.

FIG. 9b is an illustration of field use of a magnetic retrieval device.

FIG. 10a is an illustration of views of an opaque magnetic SPME device.

FIG. 10b is an illustration of views of a clear magnetic SPME device.

FIG. 11 provides charts of relative distribution of individual PAHs in sediment samples at the five MGP sites.

FIG. 12 provides a chart of SPME-LIF response for sorbent rods exposed to sediments for different times.

FIG. 13 provides a chart illustrating the effect of sample volume on SPME-LIF response after 18 and 48 hours.

FIG. 14 provides a chart illustrating the direct LIF response for pure water (A) and water with 9 mg/L of fulvic acid (B) compared to the SPME-LIF response (18 hour) for pure water (C) and 9 mg/L fulvic acid (D).

FIG. 15. Comparison of SPME-LIF response with total pore water PAH-34 toxic units (top left), total PAH-34 pore water concentrations (bottom left), total sediment PAH-34 concentrations (top right) and total sediment PAH-34 concentration on an organic carbon basis (bottom right) for the 33 surface sediments from sites A, B, C, and E.

FIG. 16 provides graphs of Spearman rank correlations for SPME-LIF responses compared to total pore water toxic units (top), total pore water PAH-34 concentrations (middle), and total sediment PAH-34 concentrations (Sites A, B, C, and E).

FIG. 17 provides graphs of comparison of SPME-LIF response with total pore water PAH-34 toxic units (top) and total pore water PAH-34 concentrations (bottom) for the 32 sediments from sites A, B, C, and E, and the 11 sediments from site D.

FIG. 18 provides a graph of relationship of LIF emission wavelength to PAH ring size for the 43 sediments (all sites) based on principal component analysis.

FIG. 19 provides graphs of comparison of SPME-LIF emission at 350 nm to the total PAH-34 pore water toxic units (top) and total pore water PAH-34 concentrations (bottom) for all sites (43 sediments).

### DETAILED DESCRIPTION

In the specification and in the claims, the terms “including” and “comprising” are open-ended terms and should be interpreted to mean “including, but not limited to . . . .” These terms encompass the more restrictive terms “consisting essentially of” and “consisting of.”

The term environmental location refers to various man-made and natural fluid handling structures such as channels, trenches, troughs, tubing, and pipelines designed to hold, direct, and transport water, slurries, and muds in plants, factories, processing facilities, and natural waterways, including underground waterways. The fluid handling structures could be contained within municipal waste water plants, for transporting produced waters from oil and gas production facilities or sewage treatment facilities, stormwater runoff drain systems, and natural waterways including urban surface waters (rivers, bays, estuaries), including the sediments of natural surface waters or groundwater.

The term sediment refers to any particulate matter that can be transported by fluid flow and which eventually is deposited as a layer of solid particles on the bed or bottom of a body of water. Sediment can be composed of particles including eroded soils, minerals, detritus, or precipitates.

The term pore water refers to the water filling the spaces between grains of sediment.

The term dissolved organic matter refers to a variety of organic substances (humic and fulvic acids) leached from plant and soil matter. This aqueous phase organic matter is considered “dissolved” since it is able to pass through a filter (filters generally range in size between 0.7 and 0.22 um).

The term substantially optically transparent refers to the ability of a solid or fluid matrix to pass the majority of light being directed into the matrix without significant directional distortion of or absorbance of the light as it passes through the matrix. The matrix may absorb or distort wavelengths of light outside the wavelengths of interest while remaining substantially optically transparent to wavelengths of interest.

The term fluid sample matrix refers to matrices that contain sufficient water to support living organisms that require saturated or near-saturated conditions including municipal waste waters, produced waters from oil and gas production, sewage, stormwater runoff, surface waters (oceans, rivers, bays, estuaries, ditches), ballast water of ships, groundwater and the pore water of these slurries, drilling muds, and sediments—either in situ or placed in sample jars.

The term bioavailability of PAHs refers to a measurement of the extent of soil/sediment/water PAHs that reaches a living organism and is available to participate in narcosis, which results in the degradation of cell membranes and can result in mild toxic effects or mortality depending upon the exposure. It is often expressed as F, where F is the fraction (<100%) of total PAH in soil/sediment that is available. (Also see, Bioavailability of Contaminants in Soils and Sediments: Process, Tools and Applications, pgs. 20-27, 2003), incorporated herein by reference.

The term flow (used as a noun) refers to a continuously moving or circulating substantially aqueous matrix such as water, slurries, and muds flowing in channels or pipelines.

The term optical fiber (or fibre) refers to a glass or plastic fiber that carries light along its length. Light is kept in the “core” of the optical fiber by total internal reflection. This causes the fiber to act as a waveguide. PAH spectroscopy typically uses fused silica (quartz) in order to pass ultra-violet wavelengths required to properly excite the PAHs and subsequently pass their fluorescence without absorbing the light.

The term aromatic hydrocarbons includes polycyclic aromatic hydrocarbons and monocyclic aromatic hydrocarbons.

The term polycyclic aromatic hydrocarbon (PAH) describes chemical compounds that consist of fused aromatic rings and do not contain heteroatoms or carry substituents and includes for example, naphthalenes, anthracenes, pyrenes, fluorenes, phenanthrenes and many others.

The term monocyclic aromatic hydrocarbon (MAH) includes for example, benzene and its derivatives including toluenes, ethyl benzenes, and xylenes (BTEX).

The term PAH-34 refers to the U.S. Environmental Protection Agency's (U.S. EPA) narcosis model which requires the measurement of 18 parent and 16 groups of alkyl polycyclic aromatic hydrocarbons (PAHs) (so-called 34 PAHs) in sediments to calculate the number of PAH toxic units (TU) available to benthic organisms. For example, the C4-alkyl naphthalenes in impacted sediment pore water contain more than 70 isomers but are counted as one of the “34” PAHs. Since the alkylated 3-ring and 4-ring PAHs have even more isomeric possibilities, the “34” PAHs measured actually represents many hundreds and possibly thousands of individual PAHs. See Hawthorne, S. B.; Grabanski, C. B.; Miller, D. J.; Kreitinger, J. P. Solid-phase microextraction measurement of parent and alkyl polycyclic aromatic hydrocarbons in milliliter sediment pore water samples and determination of K_{DOC} values. Environ. Sci. Technol. 2005, 39, 2795-2803. Hawthorne, S. B.; Miller, D. J.; Kreitinger, J. P. Measurement of ‘total’ PAH concentrations and toxic units used for estimating risk to benthic invertebrates at manufactured gas plant sites. Environ. Toxicol. Chem. 2006, 25, 287-296).

The term 2 to 6 ring PAHs refers to those PAHs which contain 2 to 6 aromatic rings.

The term “clean” levels refers to low PAH concentration or <0.1 SPME Toxic Unit PAH-34.

### A. Overview

A system and method for detecting analytes by providing a thin film layer absorbent substrate that is substantially optically transparent, with near zero autofluorescence, and that contaminants or other analytes have an affinity for, as compared to the soil, sediment, water, and/or other matrix in which they are typically present in environmental testing. This system and method is also useful for testing for any substantially aqueous analytes, such as, foods and beverages where trace aromatic hydrocarbon analysis is required. The invention is useful for testing all types of waters, in particular, those types of waters typically tested by environmental testing firms. Other than testing water occurring in sediment, the invention provides advantages for testing any water, because of the benefits of excluding non-PAH dissolved organic matter (DOM), which often gives false positive results with other testing methods.

According to certain embodiments, the absorbent material is exposed to sample matrices in or from environmental locations including water, saturated soil, slurry and sediment samples, where the suspected analytes may be present, and from which the analytes are extracted by their affinity for the thin film, so-called solid phase extraction. Water includes all the types of water that contaminants (PAHs, MAHs) are dissolved in. There are various bodies of water, e.g., pore water, surface water, storm water, process water or groundwater. Surface water includes water in ditches, rivers, lakes and oceans. Groundwater is water beneath the surface of the earth which saturates the pores and fractures of sand, gravel and rock formations. A commonly tested type of groundwater is well water.

Access to groundwater is typically gained by simple wells or piezometers (mini-wells). Recovered groundwater could be placed in a development vessel with an absorbent sampler for testing, or an absorbent sampler could be lowered into the well by a string or cable, allowed to soak for an appropriate time, and then later retrieved for testing.

Solid phase extraction (SPE) is a sorbent technique which relies on selective absorption of analyte into a solid material rather than a liquid solvent. After absorption, the analytes are typically desorbed by heat or liquid solvent extraction (removed from the SPE material) prior to analysis. Sheets, blocks, or relatively large SPE devices often have problems with releasing their analytes during extraction due to the depth of penetration into the SPE device. Subsequently, during desorption there is often “carryover” or incomplete release of the analyte. These problems are overcome by dispersing very minute quantities of SPE material onto rods of fused silica or other appropriate material. An additional benefit of the solid phase microextraction (SMPE) approach is more rapid approach to equilibrium with the matrix being analyzed since diffusion into the relatively small amount of SPE material in SPME occurs at the same rate as diffusion into the large SPE block, but it reaches equilibrium sooner since the mass is so tiny. Also, SPME is far less dependent on sample matrix size/volume—since saturation of the very minute amount of SPE material takes much less analyte than SPE (relatively large blocks or sheets). This is advantageous for in situ exposure of the SPME device in sediments—a situation where the technique is unable to stir the matrix to create exposure of large volumes of analyte to the SPME device. While carryover is not a concern for the SPME-Fluorescence approach (since analysis does not require subsequent removal of the analyte and the device is used only once), the more rapid equilibrium times are beneficial for fast analysis and results in a more cost effective solution since more samples can be tested in a given period of time. Use of the large blocks planted directly into sediments or saturated soils (as in Agar paper) is problematic in sediments since it's SPE, not SPME. Not enough volume of the sediment would get exposed to the block to achieve high enough concentrations to detect compared to SPME. (The immediate surroundings just around the SPME contain enough PAHs to reach equilibrium or detectable amounts). In addition, any absorbed analytes would be “diluted” in the relatively large volume of the block,

The SPE absorbent material is subsequently subjected to spectral analysis, such as LIF testing, as described, for example, at U.S. Pat. No. 7,015,484 for “Multi-dimensional fluorescence apparatus and method for rapid and highly sensitive quantitative analysis of mixtures” and U.S. Pat. No. 5,828,452 for “Spectroscopic system with a single converter and method for removing overlap in time of detected emissions,” both incorporated herein by reference.

Due to the near-zero autofluorescence of the absorbent material, any fluorescence detected in the LIF testing is most likely due to non-polar organic analytes extracted from the aqueous fluid sample matrix into the non-polar SPE material. Polar dissolved phase organics in the aqueous phase of the matrix are unlikely to sorb onto or into the SPE material. Further, because analytes have a high affinity for the absorbent substrate as opposed to the fluid sample matrix, the substrate has a concentrating effect on the analytes, thereby facilitating analyte detection. In cases where the sampler's autofluorescence varies or is not known to be consistent, it is useful to pre-measure the fluorescence of the sampler to determine autofluorescence of the sampler so that value can be subtracted from the value obtained after exposure to the sample.

### B. Testing of Fluid Sample Matrices

### 1. SPME Thin Films

In certain configurations, the layer of absorbent material is SPME material that is substantially optically transparent, has a near zero autofluorescence, and that analytes in the environment have a high affinity towards, as compared to water, soil, sediment, or other sample matrix. In a particular embodiment, the SPME material is a thin film of polydimethylsiloxane (PDMS), available from GE (Waterford, N.Y.) under the formula name of XE5844 and having the above-mentioned characteristics.

LIF analysis of PDMS coated in a thin film layer on a substrate exposed to a testing environment (e.g., fluid sample matrix) is useful in the detection of fluorescent contaminants or other analytes, such as PAHs, because of the near-zero autofluorescence of PDMS and the high degree of fluorescence attributed to the multiple aromatic rings found in PAHs. Further, the optically transparent nature of the PDMS enables the LIF analysis to generate accurate results. If the excitation light must pass through the substrate carrying the PDMS in order to reach it, the substrate may be non-fluorescent glass, quartz, plastic or other material transparent to the LIF excitation and corresponding emission frequencies, so that these are not affected.

The substrate includes for example, stainless steel wire, strips or rods, quartz strips or rods (fibers), Poly(methyl methacrylate) (PMMA) strips, rods or vessels, ceramic strips or wires, glass strips, rods (fibers) or vessels. The substrate further includes any supporting substrate that does not fluoresce, that contains the desired mechanical properties, will not rust, can be coated, and readily formed to the correct shape/size to allow for optical interrogation. The substrate could also have magnetic properties. Magnetic substrates are discussed further below in section B4.

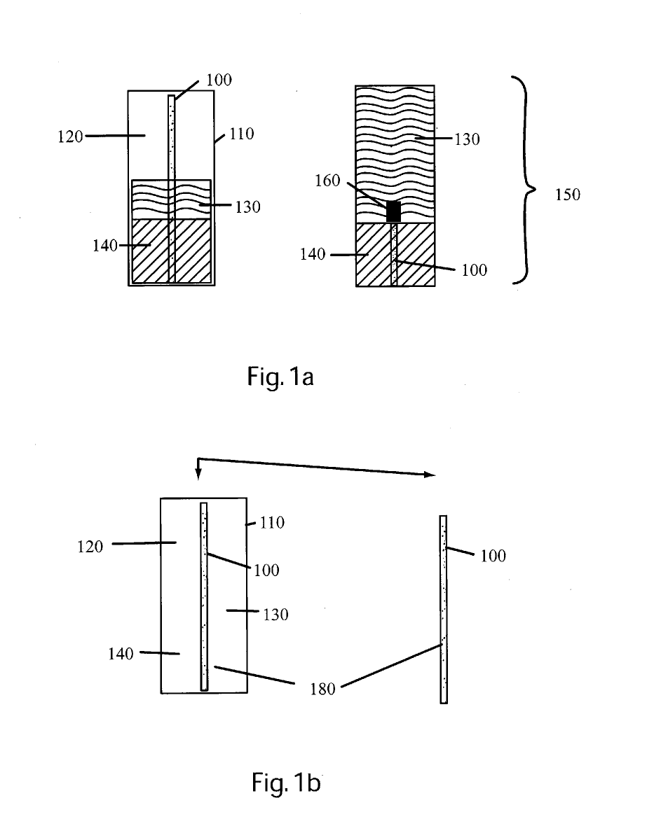

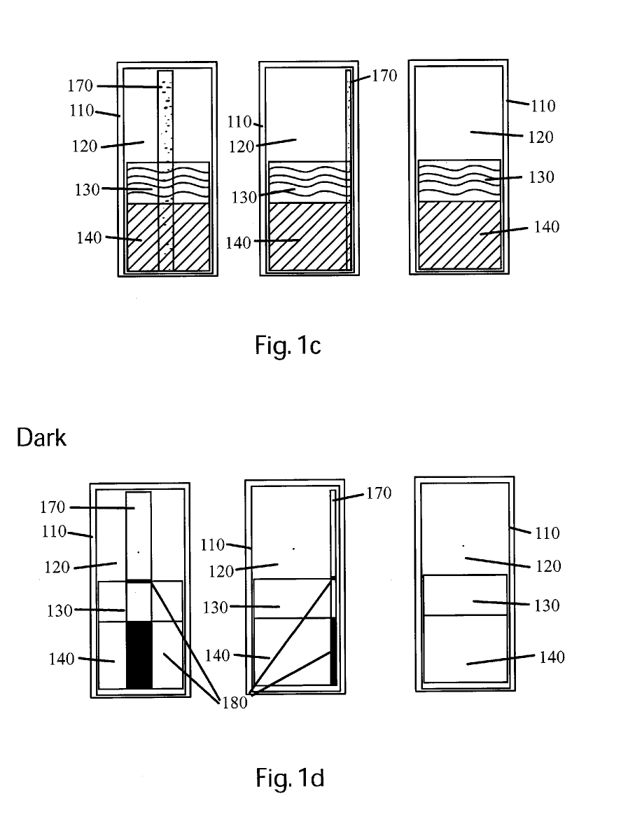

Thus, in the example of FIG. 1c (discussed below), a PDMS sampler is fabricated by coating a vessel surface with a PDMS strip. When the PDMS is supplied as a two-part system, the freshly-mixed material is a semi-viscous liquid, at least initially, and capable of being coated on substrates as a thin film. Configurations that implement viscous PDMS in a thin film involve deposition of a thin film of PDMS onto various substrates, such at jars, medicine droppers, swabs, swatches, glass tubes, fiberglass strings, optical fibers, fused silica rods, wires, silicone or Teflon® tubing or other perfluoroalkoxy (PFA), polytetrafluoroethylene (PTFE) or fluorinated ethylene propylene (FEP) tubing, and/or PFA, PTFE or FEP bags, (TEFLON is a registered trademark of E. I. du Pont de Nemours and Company) each of which may serve as substrates for collecting analytes by SPME from a fluid sample matrix.

In each of the above-mentioned configurations, PDMS thin film thickness ranges generally from about less than one micron up to about 200 microns. However, increased thicknesses are also used since appropriate film thickness is based on the analytes' solubility, mobility, and affinities toward SPE material. In some embodiments, the thickness of the

PDMS thin film layer is selected based on, inter alia, the environment to be tested, the targeted contaminants to be measured and/or detected, and/or the amount of time the test will be performed.

As described in David et al., Stir-Bar Sorptive Extraction of Trace Organic Compounds from Aqueous Matrices, LCGC North America, February 2003, sorptive extraction is by nature an equilibrium technique. For example, for water samples (substantial water content of about >10% by weight), the extraction of solutes from the aqueous phase into the PDMS extraction medium is controlled by the partitioning coefficient of the solutes between the silicone phase and the aqueous phase. In some instances, equilibrium may be achieved by controlling the thickness of the thin film deposited onto the substrate, based on a PAH's absorption rate, which may be influenced by the PAH molecular weight and the type of matrix, e.g., solid, liquid, or suspension.

For example, when testing for analytes in water environments, a PDMS thin film layer may be thicker than when testing for contaminants in below saturation (about <10% water by weight) soils. This is because, for water testing, due to its generally free flowing nature, the volume of water that comes into contact with the PDMS layer is larger than the amount of soil that comes into contact with a PDMS layer, due to the generally granular and sedentary nature of soil. In water, diffusion also assists in delivering more analyte to the layers surface than in the case of sediments, slurries or soils. Accordingly, in some configurations, as the volume of substance contacting the PDMS layer having potential for containing analytes increases, the thickness of PDMS thin film also may increase so that the PDMS layer does not become saturated in certain spots, thereby avoiding inaccurate analyte measurements. Balanced against this is the desire to reach absorption equilibrium quickly, where accurate measurement of analyte concentration requires equilibrium.

Furthermore, when particular analytes are the intended targets for testing, the thin film PDMS layer may be adjusted based on the affinity the analyte has for the PDMS, the molecule size of the analyte, and/or the expected concentration level of an analyte suspected to be present in the environment. Thus, for example, when an analyte has a high affinity for PDMS and is suspected to be present at high levels, the PDMS thin film layer is provided on a substrate at a greater thickness, so that the analyte does not create “saturation zones” that may result in inaccuracies in the measurement of the target analyte. Alternatively, when the analyte is suspected to be present at minute levels, the PDMS thin film layer is relatively thin, so that when the PDMS is analyzed after exposure to analytes, the concentration of the analyte in the thin PDMS layer will be higher compared to a thicker PDMS layer with the same exposure time. Thus, appropriate selection of thin film PDMS coatings allow rapid testing and response, because the thin film concentrates analytes in a small volume, allowing lower detection limits.

The duration of time the PDMS thin film layer is subjected to environmental testing is also a factor in selecting the thin film thickness. This is because the dwell time required for equilibrium to be reached, e.g., the time it takes for the concentration of analyte to be correlative to the analyte concentration in a thin film, varies according to factors that, inter alia, include PDMS thin film thickness. In some embodiments, by providing a PDMS thin film sampler layer, the dwell time required for equilibrium to be reached may be short (about <1 hour), and LIF testing and analysis may be performed quickly. Fast results are valuable for environmental field testing since the results can be used to determine the end of testing or need for subsequent testing prior to leaving the field. Where simple detection (simply above or below a threshold) of analytes is the desired result, as opposed to quantitative measurement, the PDMS thin film layer may be thin, because the presence of “saturation zones” is not an issue. In this case, the presence of the analyte in the PDMS is more easily detected because of the concentrating effect PDMS has on analytes, which eases the spectral equipment requirements for analyte detection. A further understanding of absorption of PAHs into PDMS and the equilibrium that effectively ends absorption may be gathered from the David et al. article cited above.

In addition to the above considerations, the thickness of the thin film PDMS layer is also dependent on inherent characteristics of the PDMS such as its bending strength when cured, and/or the thickness required for uniformly coating a development vessel. For a development vessel that may be subjected to abrasive conditions, the PDMS layer may be thicker so that has an increased durability.

2. Development Vessels with SPME Samplers

The SPME thin film of PDMS or a similar material can be deployed in certain configurations that make it more useful as an extraction and concentration tool for deriving an LIF reading for a fluid sample matrix. In one embodiment, a SPME coated substrate (rod or needle) is inserted into a water sample. FIG. 1a provides a SPME coated substrate (“sampler”) 100 inserted into a development vessel 110 containing a density-stratified sample matrix of air 120, water 130, and saturated soil, sediment or slurry 140. The sampler is inserted into the sediment at the bottom of the vessel 110 by hand or attached to the vessel's cap (not shown). FIG. 1a also shows field deployment of the sampler directly inserted into saturated soil, sediment or slurry 140 at the bottom of a water body 150. The sampler 100 is shown attached to a handle, float or tether 160.

The SPME coated substrate (“sampler”) 100 inserted into a development vessel 110 of FIG. 1a is also shown in the dark (during an analysis phase), as FIG. 1b. FIG. 1b shows fluorescence (light) 180 emitted by PAHs. The sampler 100 is shown while still in the vessel 110 under UV light, and as the sampler under UV light after it is removed from the sediment 140.

Once the PDMS layer is exposed to the saturated soil, sediment or slurry and water, a distribution of analytes in both the saturated soil, sediment or slurry and water layers may be determined through spectral analysis of the PDMS coating by testing multiple points on the PDMS strip corresponding to one or more of the air 120, water 130, or saturated soil, sediment or slurry 140 portions of the sample matrix. This embodiment is useful in environmental testing applications where it is desirable to understand the spatial distribution of contaminants, i.e., where contaminants, such as PAHs, concentrate in the environment. For example, if a light non-aqueous phase liquid (LNAPL) exists in the soil, sediment, and water slurry, then LNAPL will form a sheen at the water/air interface. This sheen will supply a flux of PAHs into the coating at a higher rate and will ultimately achieve both a higher concentration and a differing PAH size distribution than the dissolved phase PAHs in the water portion. This is due to the substantially higher solubility of PAHs in the organic solvent that makes up the bulk of the NAPL than in water (1,000 to 1,000,000 fold higher solubility in octanol vs. water (octanol/water solubility coefficients (K_{ow}s) of 3 to 6). The K_{ow} generally increases with increasing ring count (size) of the PAH. This differential fluorescence response will result in a “band” of fluorescence at the interface, indicating sheen or presence of LNAPL. This information is of great use to investigators as the presence of the LNAPL indicates that this sample location has “source term”—a phrase used to denote that the LNAPL can feed PAHs into the aqueous environment since it acts as a “stockpile” or source of PAHs. The presence of sheen or source term is often difficult to determine visually since sediments, soils, and water samples often mask the presence. Oil soluble indicator dyes such as Sudan Red have been used to “highlight” NAPL for visual detection, but detection limits are often higher than desired and/or results are inconclusive. In addition, iron nitrifying bacteria can create a sheen that is often mistaken for LNAPL. This sheen, even if present, would not transfer differential amounts of PAHs into the PDMS strip and would therefore not provide a “false positive” for sheen.

In another embodiment, the thin film is made part of a vessel in which a sample may be placed, developed and analyzed. FIG. 1c provides a PDMS-coated sampler strip 170 placed as a vertical stripe on an interior surface in a development vessel 110 that contains a density-stratified sample matrix of air 120, water 130, and saturated soil, sediment or slurry 140 of a sample introduced as a mixture. The transparent vessel 110 is shown as a front view, side view and as a vessel without a strip. Here, development is based on the operation of gravity to provide density separation.

In one embodiment, the development vessel 110 is optically transparent and does not fluoresce at all. This permits visual observation, but. more important, the LIF excitation light applied to the sampler strip 170 can be directed from, and the corresponding emission light detected from, the vessel exterior. This can be done at any selected point on the strip 170 to detect analytes captured at that point from the air 120, water 130, or saturated soil, sediment or slurry 140. LIF may not result in the contaminants visibly fluorescing at all locations on the strip 170 (as suggested by the figure), because the contaminants are present in trace amounts in the sample matrix and the SPME can improve concentration only to the extent of the partitioning coefficient(s). However, with known detectors, the emitted light over a spectral range of interest can be detected. In order to prevent the fluorescence analysis from penetrating through the optically clear film and analyzing fluorescent materials inside the jar directly, a thin backing film of opaque non-fluorescent silicone or other opaque but analyte soluble material could be applied to the inside surface of the SPME film to assure that the analysis only measures what is in the SPME thin film, and not what is behind it (sample in the vessel).

FIG. 1c is also shown in the dark (during an analysis phase), as FIG. 1d. FIG. 1d shows fluorescence (light) 180 emitted by PAHs. FIG. 1d shows the samplers while still in the vessels 110 as a front view, side view and as a vessel without a strip.

One would observe that the samplers reveal that fluorescing analytes in the sample matrix are present: in the highest concentration in water, where the shading of the strip is the lightest; at zero concentration in air, where the shading of the strip is the darkest; and in an intermediate concentration in saturated soil, sediment or slurry, where the shading of the strip falls between the highest and zero concentrations. In addition, the portion of the sampler corresponding to the water layer would show a gradation of contaminants in which the top segment of the water-exposed portion of the sampler has the highest amount of analytes, corresponding to the lightest shade segment, at the air/water boundary.

In the above-described embodiment, one or multiple points of the samplers may be analyzed using spectral analysis, such as LIF or fluorescence. Raman and absorbance spectroscopy may also be utilized, depending on concentrations and analyte's spectral behavior. While LIF will result in lowest detection limits and greatest sensitivity due to benefits of laser excitation, cheaper and more readily available spectroscopic techniques might be used such as light-emitting diode and lamp-based fluorometers used in chemistry and life science markets (high throughput screening, genomics, and proteomics). Fluorometers that are able to excite the PAHs at the correct wavelengths and measure the resulting fluorescence would be capable of reading the samplers.

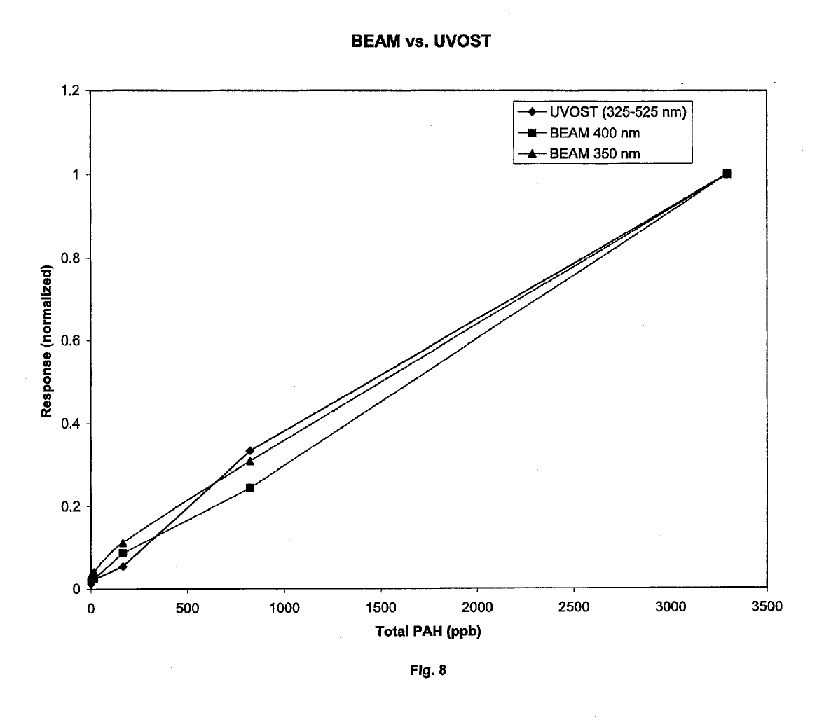

FIG. 8. shows graphically that SPME-F testing (fluorometer testing) scales with SPME-LIF (laser induced fluorescence testing). Total PAH responses were compared using either a BEAM handheld fluorometer (modified with an alternate excitation wavelength LED and emission filters) available from Dakota Technologies, Inc (Fargo, N. Dak.) for SPME-F testing, or a UVOST® fluorometer available from Dakota Technologies, Inc (Fargo, N. Dak.) for SPME-LIF testing.

In the embodiments shown and described with LIF, it should be understood that LIF is subset of fluorescence. Other fluorescence analysis such as non-laser excitation with LED, or lamps are a cost effective alternative to LIF testing.

Use of a clear development vessel with an interior surface coated with a thin film of PDMS allows spectral analysis of the analytes sorbed into the PDMS without handling or disturbing the PDMS layer for optical analysis. However, the PDMS strip 170 might also be made in the form of a thin film coated “dip stick”, dropper or needle which is attached to the cap or lid of the vessel 110 and left to hang from above down into the sample matrix, but made removable for testing.

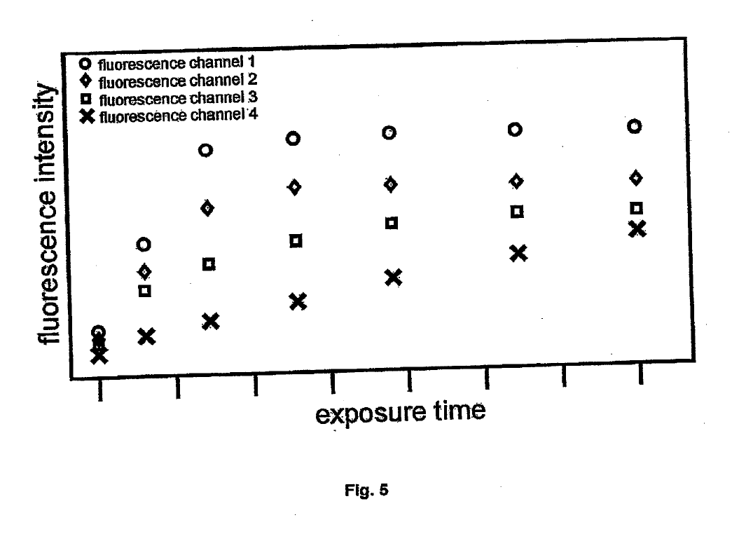

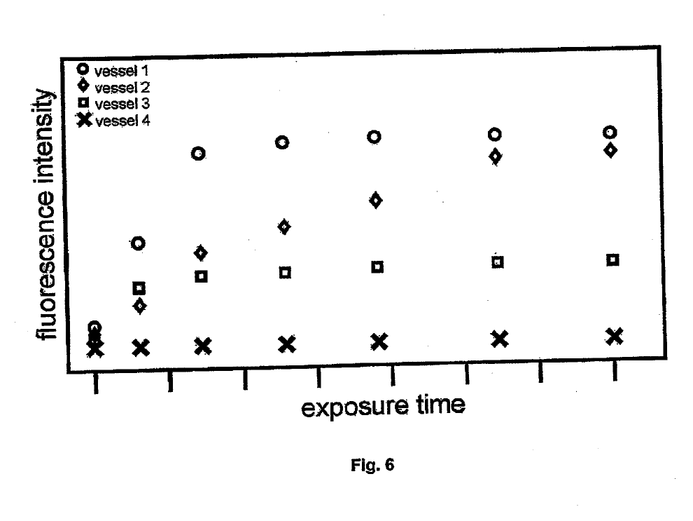

In another embodiment, the PDMS thin film layer exposed to the matrix for a period of time is subjected to repeated LIF or fluorescence analysis at a single point on the PDMS thin film layer. Such LIF analysis begins as soon as the sample matrix is placed in the vessel, so that data representing the progression of absorption into the PDMS layer can be tracked. The LIF system can include means for fast capture of multiple spectra in a time sequence of LIF readings stored for later display and analysis. The result of each spectral analysis may appear in the form of a graph, such as the set of curves shown in FIGS. 5 and 6. As can be seen, when the equilibrium level of absorption is approached, the intensity values slow their increase (circles first—X's not yet). This may occur at different rates in different parts of the spectrum, because the different parts of the spectrum are characteristic of different analytes. FIG. 5 shows an example of results from one SPME device in one sample. The different fluorescence channels represent different size PAHs. The size of PAHs generally determines the speed at which they move into and come to equilibrium in the SPME device.

FIG. 6 shows an example of results for SPME devices from a plurality of vessels in a carousel. Samples of each vessel come to equilibrium at different points in time or at different intensities which point to differing PAH concentrations or size distributions. The carousel is discussed further below.

These time sequence of intensity data curves can reveal a variety of information. By showing the approach of equilibrium, the portions of the curve showing little further intensity increase can signal when a final reading should take place and the LIF/fluorescence analysis resources can be moved to a different area of a strip 170 or moved to a totally different sample. The determination of when a sufficient equilibrium is reached is simplest when only a single analyte or class of analytes, e.g., total PAHs, is of interest. Where more than one analyte is involved and the equilibrium for each analyte is approached at different rates, the determination is more complex. The intensity value at equilibrium may be properly scaled and calibrated to yield a value that is representative of the concentration of the analyte in the sample matrix. Various approaches to calibration of systems are commonly used in analytical chemistry. A reference emitter is a known substance that fluoresces very consistently and is stable over time. For example, a fluorescent plastic rod, the same shape/size of the sampler, which can be analyzed just prior to analyzing the actual sampler. The actual sampler value is then normalized by the response of the reference emitter and data is related in terms of RE (% RE). Historical data, also measured with respect to the same RE, is then used to “estimate” the aromatic hydrocarbon content. An estimation is possible if, after many measurements on a variety of systems, there is a consistent relationship between % RE and Total PAH by other analyses. In this manner in situ samples could be quantified since no sample is actually “recovered”—just the sampler. One could also analyze a subset of recovered sediment samples by SPME-F, then send that subset of RE-quantified recovered samples on to be further analyzed by SPME-GCMS or other analyses. A correction factor based on the lab results could then be applied to the entire set to normalize them to the lab results. Other viable methods for calibrating discrete samples would include addition of known quantities (spiking) with water soluble internal standards and/or the method of additions. Thus, fluorescent analysis provides not only for the detection but also concentration measurement of analytes in the fluid sample matrix.

For example, in the development vessel 110 of FIG. 1c, a point on the PDMS sampler strip 170 in the pore water layer 140 may be read multiple times by LIF over a period of time in order to determine the time at which an analyte of interest there has reached equilibrium. Once equilibrium for a given analyte present in the sampler layer subjected to LIF has been reached, a concentration measure for the analyte detected there is then developed.

The above-mentioned development vessel 110 may also be subjected to LIF analysis at a single point using differing excitation wavelengths over a period of time in order to detect the presence of multiple PAH classes in the matrix. It is useful to show the presence of various PAHs, which have differing excitation energies, and in some instances, differing absorption rates. Thus, one PAH has a maximum excitation energy of 275 nm, e.g., naphthalene, and another has a maximum excitation energy of 350 nm, e.g. pyrene. By conducting LIF with the sampler 100 being exposed to various wavelengths, multiple PAH classes could more readily be detected and differentiated in a matrix. Furthermore, by conducting LIF at various wavelengths multiple times over a sufficient time period, PAHs having a slow absorption rate (not just those first reaching equilibrium) may be detected and/or measured by LIF analysis. Moreover, by conducting LIF using differing excitation wavelengths over an observation period, the amount of time for various PAHs to reach equilibrium may be determined, which may help to identify the class of PAH or its NAPLs source. For instance, advanced analysis could determine that the PAH source NAPL is coal tar, rather than diesel fuel.

In yet another example, the above-mentioned development vessel 110 is subjected to LIF at various points along the PDMS layer 170, e.g., at points on the PDMS thin film layer corresponding to where the water 130 and saturated soil, sediment or slurry 140 layers contact the PDMS thin film layer; and the LIF may be conducted multiple times over a period of time using differing excitation wavelengths in order to determine the spatial distribution and concentration of different PAHs in the matrix.

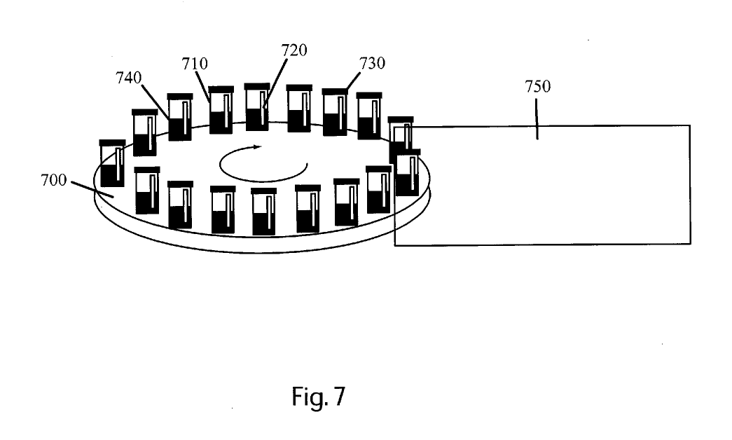

In a further embodiment, a number of development vessels, e.g., 17 development vessels 710, which also have caps 730, are each coated with a PDMS thin film sampler layer 720 on the side of each vessel's interior are partially submerged in a fluid sample matrix 740, and are provided on a carousel 700 as shown in FIG. 7. The 17 development vessels are shown for illustration purposes only, and any number of development vessels are contemplated, e.g. 50 or more. This embodiment helps to measure analytes at higher throughput rate than would be achievable by manual procedures. The carousel 700 is rotated into position by a fluorescence analyzer 750 for testing of the contents of each development vessel 710. The use of a carousel would allow the technicians to focus on preparation of samples and data analysis rather than the laborious procedure of loading and unloading a large number of samples for which analysis is desired.

Because LIF excitation/reading cycles can be done in seconds (or less), the use of multiple development vessels enables the real time detection and/or measurement of changing analytes in a matrix. For example, for PAHs having a fast absorption rate, detection of a PAH in real time or near real time is possible. Measurement of PAHs in a fluid sample matrix may also be possible in real time or near real time in some configurations when PAH equilibrium can be reached after a short exposure time (about 1 to 2 hours) to the PDMS strip 100.

3. Transportable SPME Recording Media

In another embodiment useful for detecting or measuring analytes in an environment that changes over time, such as a flow that may vary as to its constituents, fiberglass, metal wire, fused silica fibers, or tape having a thin film coating of PDMS or other thin, coatable SPME material, forms an SPME recording medium stored on, for example, a spool or in a cassette, and has segments selectively transported into and out of a testing environment, such as a stream of running water, drilling mud, or sewage with dissolved or entrained analytes.

An example of where this would be useful is for monitoring oil production produced waste water or urban runoff for PAH concentration continuously and in real time. The SPE-fluorescence system would report when PAH concentrations have exceeded certain concentrations, signaling transfer of the contaminated waters to treatment facilities and/or holding basins. Once PAH concentrations return to “clean” levels, the waters could then be re-routed back into the normal discharge receptor (bays, river, and lakes). This is valuable for preventing high PAH concentrations from building up in our nations rivers, bays, and lakes. For example, a PDMS coated strand is periodically drawn through a fluid medium and subsequently drawn into an analyzer for LIF analysis. The transport rate of the coated strand may be controlled to provide a selected dwell time for the presence in the flow of any given point. The transport rate may also be controlled based on an analysis of the dwell time required to reach sufficient fluorescence signal levels and/or equilibrium. This embodiment would also be useful for monitoring for dissolved phase PAHs or NAPL sheen in a river during dredging or other projects upstream of the monitoring point to determine if contamination is being released and getting past controls meant to minimize their release during remediation of PAH impacted sites such as former manufactured gas plants (coal tar).

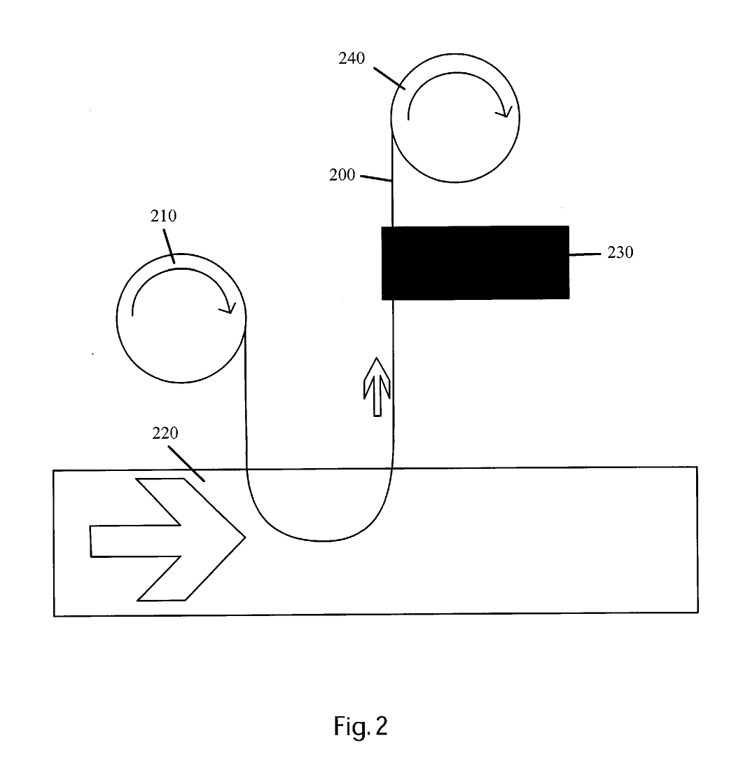

FIG. 2 provides an illustration of an SPME coated wire/string/filament 200 fed from spool 210 of stored coated filament into an aqueous or other flow environment (matrix) 220 where wire/string/filament 200 is exposed to changing contaminant concentrations and the SPME coating absorbs contaminants (PAHs) having an affinity for the SPME material over the environmental elements such as sludge, mud, and water. In some configurations, the wire/string 200 is drawn through the environment 220 and after its exit subjected to fluorescence, or other analysis, via optical analyzer 230 in order to detect the presence of analytes. The wire/string 200 may be drawn through the matrix 220, and into analyzer 230 at a constant rate, for example, so that the presence of contaminants or other analytes in the environment may be continuously monitored. The spent coated wire/string/filament is collected on spool 240.

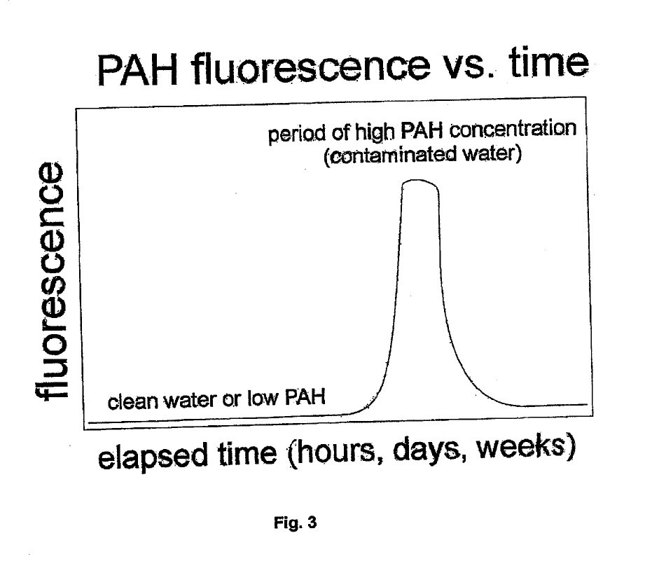

FIG. 3 is an illustration of the result of continuous monitoring of PAHs in a flow or other changing environment, presented as a graph showing a log of PAH concentration (measured by fluorescence intensity) vs. time. This technique enables the monitoring of contaminants or other analytes over extended periods of time (e.g., hours, days, weeks), which is useful, for example, in tracking the progress of clean-up or “polishing” process used to remove PAHs.

Feeding wire, fiberglass, string or other transportable SPME recording media through aqueous media is useful in applications that monitor PAHs from sources such as in tar, grease and tire residue found in storm water, bays, harbors, rivers, and pipes. The recording media is passed through the aqueous media, with the fresh surfaces providing new time record data that may be presented to the LIF analyzer continuously. With appropriate selection of the dwell time in the aqueous media (or other flow), the use of PDMS coated materials continuously passing through a LIF analyzer allows for lower detection limits orders of magnitude lower than direct measurement, because PAHs concentrate into the PDMS matrix, the PDMS isolates the PAHs from the turbid/cloudy analyte matrix (which reduces optically interrogated volume), and preferentially absorbs PAHs vs. natural occurring dissolved humic and fulvic acid false positives, making it ideal for spectral analysis. In view of the above advantages, it is possible to create an automated system capable of providing real-time, continuing results of PAH analysis, and in some instances, a system capable of quickly detecting, and responding to the detection of, PAH contaminants. This advantage also reduces the requirement of humans traveling to drainage sites to test runoff samples, which can be expensive, when it is desired to collect hundreds of samples over a monitoring period.

In an alternative embodiment, the wire/string recording medium 200 is exposed to aqueous environment 220 for a dwell time sufficient for equilibrium to be reached, and then subjected to LIF or other analysis, in order not only to detect but to measure the concentration of contaminants present in aqueous environment 220. The dwell time is based on the thickness of the thin PDMS layer, the particular analyte and solutes and other factors that affect the equilibrium process. For example, the thickness of the PDMS thin-film layer deposited on the wire/string 200 is calibrated so that equilibrium for a particular target PAH is obtained after a predetermined wire/string 200 dwell time.

### 4. SPME Coated Magnetic Substrates

In another embodiment, SPME coated substrates are magnetic, in particular, a magnetic substrate that resists corrosion. For example, a magnetizable or magnetic stainless steel substrate that is coated with SPME material. Magnetic stainless steel is commercially available with varying alloys to give stronger or weaker magnetic properties to the stainless steel. All stainless steels, with the exception of the austenitic group, are strongly attracted to a magnet.

The magnetic SPME coated substrates are retrieved from vessels or flows with magnetic retrieval devices. This method provides a convenient way for a user to retrieve the SPME coated substrate from various water environments. The magnetic retrieval device is either permanently or removably connected to the SPME coated substrate. Magnetic retrieval can be performed with a device, typically rod shaped, used in the laboratory for retrieving magnetic objects, e.g. from beakers. In the field, magnetic retrieval devices are typically attached to a line/wire or rod so that the user can retrieve magnetic objects from a boat or from a shoreline.

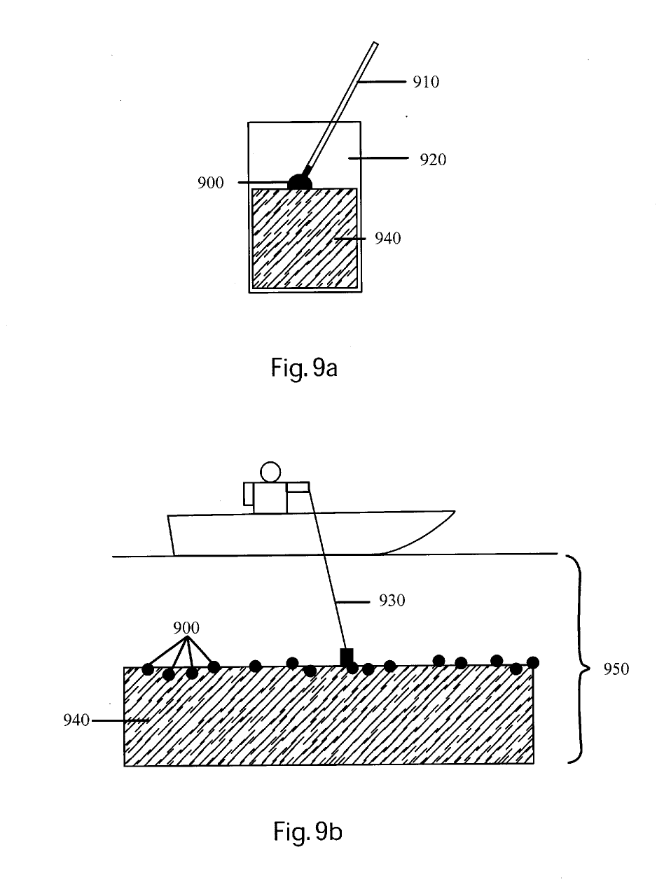

FIG. 9a provides an illustration of retrieval of a magnetic SPME coated substrate 900 with an attached magnetic retrieval device 910, which is rod shaped, from a vessel 920. The SPME coated substrate 900 is shown placed into the matrix containing saturated soil/sediment/or slurry 940. The SPME coated substrate 900 is shown at the top of the sediment for ease of viewing and will sink or can be inserted fully into the sediment. The magnetic retrieval device 910 is useful to retrieve SPME coated substrate 900 that has penetrated down into the sediment and is no longer viewable.

FIG. 9b provides an illustration of retrieval of a magnetic SPME coated substrate 900 with an attached magnetic retrieval device 930 from a body of water 950. The SPME coated substrate(s) 900 are shown placed into the top of a matrix containing saturated soil/sediment/or slurry 940 within the body of water 930. Some SPME coated substrates 900 are shown at the top of the sediment for ease of viewing but will also sink into the sediment with the aid of gravity. The SPME coated substrates 900 covered with sediment are retrieved by probing the sediment with the magnetic retrieval device 930 until magnetic contact is made with the substrate for reattachment and retrieval. This embodiment would allow broadcast spreading of samplers over large areas (bays, harbors, rivers, shorelines). Subsequent retrieval and analysis of a potentially limitless number of samplers would allow investigators to determine PAH distribution at previously unobtainable detail with great efficiency. Since stainless steel and PDMS are extremely inert (and used routinely in implants in humans) regulations should allow those devices not retrieved to be left in place.

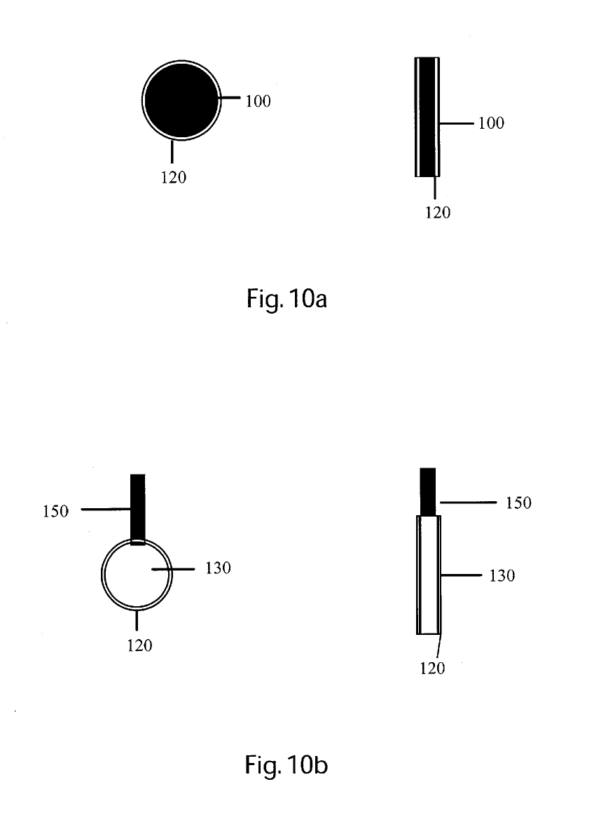

FIG. 10a provides an illustration of magnetically susceptible substrates 100, 110 with a SPME coating 120 on the outside of each substrate. The substrates, could be any shape, in particular the substrates are shaped for ease of coating, e.g., round or rod shaped. With opaque substrates, optical interrogation of both sides of the SPME coated substrates is not done at once. The fluorescence signals are half of that obtained with clear substrates.

Adjustments can be made in filtering of the expected increase in excitation light scattering that would result. However, there is no major effect on performance because it is the background fluorescence that limits performance not the absolute flux of fluorescence from the coated magnetically susceptible substrates.

FIG. 10b provides an illustration of substantially optically clear substrates 130, 140, with a SPME coating 120 on the outside of each substrate. A magnetically susceptible “handle” 150 is either permanently or removably attached to the coated substantially optically clear substrates 130, 140.

### 5. Methods of Analyzing

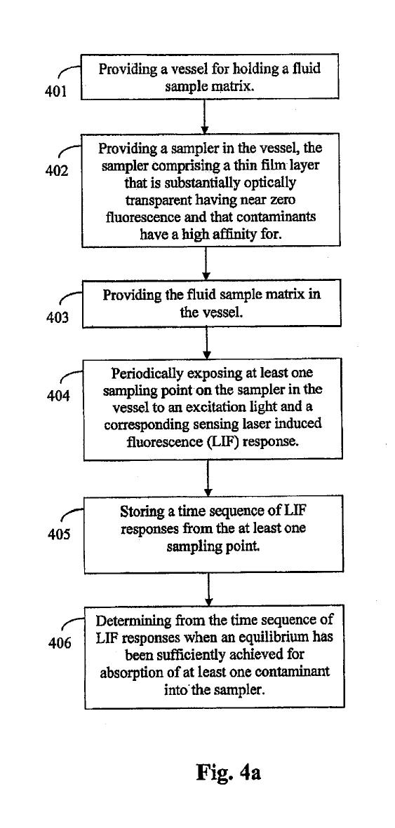

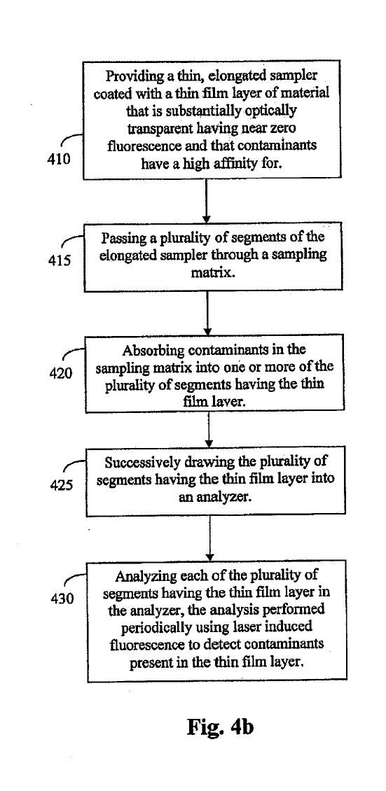

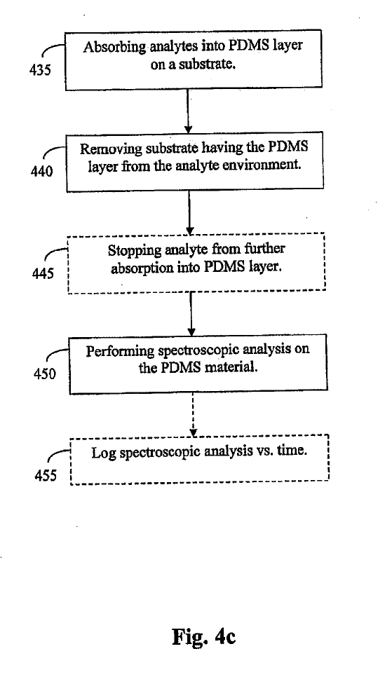

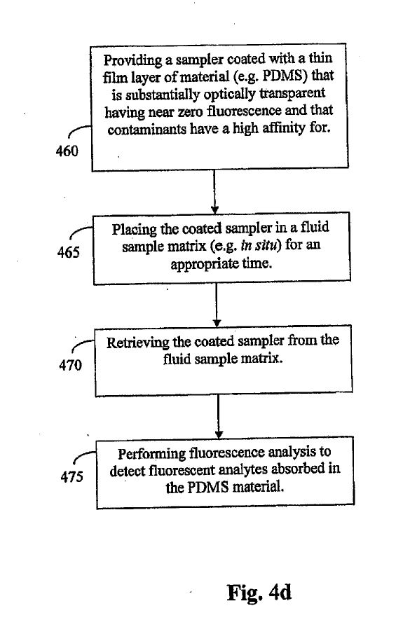

FIG. 4a provides a flowchart of a method for analyzing trace contaminants present in a sample matrix. According to FIG. 4a, the method for analyzing trace contaminants present in a sample matrix includes, providing (401) a vessel for holding a fluid sample matrix, providing (402) a sampler in the vessel where the sampler is composed of a thin film layer that is substantially optically transparent and has near-zero autofluorescence and for which at least one contaminant has a high affinity. A fluid sample matrix is provided (403) in the vessel, and at least one sampling point on the sampler in the vessel is periodically exposed (404) to an excitation light followed by sensing of a corresponding fluorescence response. A time sequence of LIF responses from the at least one sampling point may be stored (405), and a determination (406) is made from the time sequence of LIF responses when an equilibrium has been sufficiently achieved for absorption of at least one contaminant into the sampler.

For example, when the rate of increase of an intensity measurement falls below a threshold value. In an alternative embodiment, a fluorometer, a fluorescence instrument that does not utilize laser induced fluorescence can be used.

FIG. 4b provides another flowchart of a method for analyzing trace contaminants present in a sample matrix. According to FIG. 4b, the method for analyzing trace contaminants present in a sample matrix includes providing (410) a thin, elongated sampler coated with a thin film layer of material that is substantially optically transparent and has near-zero auto fluorescence and for which contaminants have a high affinity, and passing (415) a plurality of segments of the elongated sampler through a sampling matrix where contaminants are absorbed (420) from the sampling matrix into one or more of the plurality of segments having the thin film layer. A plurality of segments having the thin film layer are successively drawn (425) into an analyzer, and each of the segments having the thin film layer is analyzed (430) in the analyzer, where the analysis performed periodically using laser induced fluorescence to detect contaminants present in the thin film layer.

FIG. 4c provides a flowchart of a method for detecting the presence of and/or measuring the amount of contaminants, according to certain implementations. According to FIG. 4c, analytes such as contaminants are absorbed (435) into a PDMS layer disposed on a substrate and the substrate with the PDMS layer is removed (440) from the sample matrix environment containing the analytes. Further absorption of the analyte may optionally be stopped (445), for example by rinsing the development vessel to clear any remaining debris, and spectral analysis is performed (450) on the PDMS material. Spectral analysis yields results such as analyte presence and, in some instances, measurement of the amount of the analytes present in the environment. In addition, logging (455) of the results of the spectral analysis over time may optionally be provided, which may yield results similar to the graph provided in FIG. 3.

FIG. 4d provides a flowchart of a method for analyzing trace contaminants present in a fluid sample matrix in situ. According to FIG. 4d, a sampler is provided that is coated with a thin film layer of an SPME material (e.g. PDMS) that is substantially optically transparent having near zero fluorescence which contaminants have a high affinity for (460), the coated sampler is placed in a fluid sample matrix (e.g. in situ) for an appropriate time (465), the coated sampler is then retrieved from the fluid sample matrix (470), and fluorescence analysis is performed to detect fluorescent analytes absorbed in the PDMS material. The “appropriate” time in step (465) could be when equilibrium occurs, a fixed and known dwell time that have been established, e.g., 18 hours, or could be a time that is less than the time taken to reach equilibrium. It is advantageous to have a simple procedure which provides consistent enough performance to provide field screening information with relative ease of use. There will be cases where the user does not want to wait for equilibrium to be verified—instead it is assumed—knowing that occasionally it has not happened. However, the fixed dwell time accounts for knowing that equilibrium has not yet occurred.

While the above methods as shown in FIGS. 4a, 4b, 4c and 4d speak in terms of removing the PDMS from the sample matrix, as an alternative, the fluid sample matrix may be removed from contact with the PDMS (as by rinsing) or, with the PDMS placed on an optically transparent substrate, the fluorescence or LIF analysis can occur without separating the PDMS and the sample matrix, as long as it is recognized that any absorption process not at equilibrium may continue to change the intensity readings obtained from a fluorometer or LIF. Further, although the methods are described as applied to contaminants (PAHs), the are equally applicable for any analyte detectable by LIF or fluorescence.

Fluorescence analysis of PAHs present in PDMS provides advantages over other types of analysis of PAHs because fluorescence analysis is non-destructive, enabling a PDMS sampler to be further analyzed via other spectral methods or successively obtaining LIF readings, if desired. It also allows the sampler to be placed back into the sample for further absorption. The time fluoresce analysis takes also is relatively short, e.g., a few seconds per sample (or even less, with a specialized data collection chip, such as shown in U.S. Pat. No. 6,816,102 for “System for digitizing transient signals” or U.S. Pat. No. 6,975,251 for “System for digitizing transient signals with waveform accumulator”), both incorporated herein by reference, and the analysis results are correspondingly fast. This enables numerous samples to be analyzed over a short period of time. In addition, because of its non-destructive nature, fluorescence analysis of the sampler can be essentially continuous, so that analysis of various points along a length of an exposed sampler may be logged. Alternatively, high-rate sampling of one point (or multiple points) over time using fluorescence analysis is performed. LIF also may provide specific results related to the detection of an aromatic class of contaminants, such as PAHs, e.g., petroleum and petroleum-based contaminants.

Furthermore, SPME-LIF or SPME-F methods are non-depletive of the PAHs in the original sample, which allows subsequent analysis of the same sample via other non-destructive, or even destructive techniques, e.g., solvent extraction or thermal desorption. The necessity for subsequent testing of the sampled location could be indicated based on the results obtained from the SPME-LIF or SPME-F testing.

Although LIF analysis of PDMS thin film layers has been the focus of the above discussion, other spectral analysis of PDMS or other SPME thin film layers are also be suitable according to certain implementations if the analytes and SPE material are amenable to these methods and suitable limits of detection are attainable.

### C. In Situ Determination of PAHs in Sediment Pore Water

In situ sampling with solid-phase microextraction (SPME) was coupled with laser induced fluorescence (LIF) to provide a simple field-portable method to determine total dissolved PAH (polycyclic aromatic hydrocarbon) concentrations in sediment pore water.

Bioavailability of PAHs refers to a measurement of the extent of soil/sediment PAH that reaches a living organism and is available to participate in narcosis, which results in the degradation of cell membranes and can result in mild toxic effects or mortality depending upon the exposure. It is often expressed as F, where F is the fraction (<100%) of total PAH in soil/sediment that is available. (Also see, Bioavailability of Contaminants in Soils and Sediments: Process, Tools and Applications, pgs. 20-27, 2003), incorporated herein by reference. Pore water PAHs are a major source of exposure for living organisms. Studies have shown that even in the presence of other forms of contamination such as PAH-containing NAPL or sorbed PAHs, the pore water dissolved PAH's strongly predict toxicity and are therefore believed to dominate as the bioavailable PAH fraction available to living organisms. The methods described below are useful for testing for PAHs including bioavailable PAHs.

PAHs can enter into and disrupt functions within living organisms, for example, PAHs that are dissolved in an aqueous phase. Having a method that quickly and easily detects dissolved PAHs provides a useful tool to estimate in situ total PAH pore water concentrations in the field, for risk assessment. If PAHs are sorbed or strongly locked onto, for example, carbon, mineral particles, or other detritus, then those PAHs are bound and do not absorb into the membranes of living organisms from an aqueous phase.

Advantageously, the SPME-LIF approach can be used on-site to rapidly map the relative PAH pore water concentrations, and those results could be used to select sampling areas for more complete testing such as pore water PAH-34 by GC/MS and biological toxicity studies. The coated rods are inexpensive and the LIF measurement requires only a few minutes per sample using instrumentation similar to that already routinely deployed in field studies. In addition, no solvents or other hazardous materials are needed to perform SPME-LIF testing in the field, eliminating the production of hazardous waste commonly associated with solvent extraction methods.

Separation of dissolved organic matter from pore water is not practical in the field. However, since DOM is polar and has high water solubility, non-polar sorbents used for in situ pore water sampling (e.g., PDMS) should largely exclude DOM, while collecting the non-polar PAHs.

PAH contamination is common in sediments near former manufactured gas plants, coking facilities, wood-treating facilities or large urban areas with major anthropogenic sources of PAHs such as combustion engines and tire wear. The water bodies adjacent to such sources often contain tremendous amounts of living or dead vegetation (reeds, peat moss, detritus, etc.). These break down in time and convert to humin, humic acids, fulvic acids, and a wide variety of naturally fluorescent materials. It is common for many sediment samples to contain significant plant material and for coal tars, creosotes and other PAH NAPLs to exist in amongst the layers of plant material. Sometimes layers of these materials actually act as the “conduits” for their transport and/or sponges that absorb and hold NAPLs. The pore water in between the sediment grains, even if the sediment itself is free of actual plant material, can have significant concentrations of colored (fluorescent)-dissolved organic matter (DOM). Any technique relying on fluorescence will need to account for their presence or minimize their impact by somehow rejecting fluorescence produced by DOM. The SPE materials such as PDMS excel at absorbing hydrophobic non-polar hydrocarbons while at the same time remaining very poor absorbers of the polar water-dissolved phase humic and fulvic acids. Finally, humic and fulvic acids are known quenchers of fluorescence (M. U. Kumke, H.-G. Löhmannsröben, Th. Roch, Fluorescence quenching of polycyclic aromatic compounds by humic acid, Analyst, 1994, 119, 997-1001) so isolating the PAHs away from potential quenchers yields even higher fluorescence response as when they co-exist. This “matrix isolation” of PAHs away from fluorescent and PAH-quenching polar DOMs is a key advantage of this SPE-Fluoresence method.

### 1. Summary of Method for In Situ Testing

Fused silica rods with a 50 μm coating of optically-clear polydimethylsiloxane (PDMS) were inserted directly into sediment/water slurries. After one to 140 hours (typically 18 hours), the coated rods were recovered, rinsed with water, and their LIF response was measured with excitation wavelength (308 nm) and emission wavelengths (350 to 500 nm) chosen to monitor 2- to 6-ring PAHs. Although a rod shape was used for testing, other shapes are useful. The rod shape is readily inserted into and removed from sediments with little abrasion of the PDMS and/or force required. In addition, a convenient holder for rods is readily constructed and repeatable positioning was accomplished for multiple reading events over time. The rod shape, in particular, was simply convenient for handling purposes.

SPME rods were selected that had low intrinsic fluorescence background and rapidly approached equilibrium with the pore water. Four emission wavelengths associated with 2- to 6-ring PAHs were monitored and the emission intensities were compared to pore water and sediment concentrations of the PAH-34, and the total PAH “toxic units” (TUs) calculated using the EPA hydrocarbon narcosis model, (U.S. Environmental Protection Agency. Procedures for the derivation of ESBs for the protection of benthic organisms: PAH mixtures; EPA/600/R-02/013; Office of Research and Development: Washington, D.C., 2003), incorporated herein by reference.

SPME-LIF response was independent of sediment sample size, as is required for equilibrium sampling methods to be used in situ in the field. Potential interferences from high and variable background fluorescence from dissolved organic matter were eliminated by the use of the non-polar PDMS sorbent. The detection limit in pore water was about 2 ng/ml (as total PAH-34), which corresponds to about 0.2 EPA PAH toxic units.

Good quantitative agreement (r²=0.96) for total PAH-34 pore water concentrations with conventional GC/MS determinations was obtained for 33 surface sediments collected from former manufactured gas plant (MGP) and related sites. Quantitative agreement between SPME-LIF and GC/MS total PAH-34 concentrations was also good for 11 sediment cores (r²=0.87), but the predominance of two-ring PAHs (compared to the other sites) resulted in a lower relative SPME-LIF response compared to the surface sediment samples. The method is very simple to perform, and is directly applicable to field surveys.

### 2. Sediment Collection and Characterization for GC/MS Testing

Sediment collection procedures and analytical methods have been described in detail in journal articles. (Hawthorne, S. B.; Grabanski, C. B.; Miller, D. J. Measured partitioning coefficients for parent and alkyl polycyclic aromatic hydrocarbons in 114 historically contaminated sediments: Part 1. K_{CO} values. Environ. Toxicol. Chem. 2006, 25, 2901-2911. Hawthorne, S. B.; Grabanski, C. B.; Miller, D. J.; Kreitinger, J. P. Solid-phase microextraction measurement of parent and alkyl polycyclic aromatic hydrocarbons in milliliter sediment pore water samples and determination of K_{DOC} values. Environ. Sci. Technol. 2005, 39, 2795-2803. Hawthorne, S. B.; Miller, D. J.; Kreitinger, J. P. Measurement of ‘total’ PAH concentrations and toxic units used for estimating risk to benthic invertebrates at manufactured gas plant sites. Environ. Toxicol. Chem. 2006, 25, 287-296,) all incorporated herein by reference.

In brief, sediments were collected using a Ponar grab sampler or, for the subsurface samples (Site D, described below), using 3-inch Vibracores. Vibracores are a cylindrical sampling tube that is vibronically delivered into the sediment with a vibrating drive head at the top of the core, driving the sediment up into the cylindrical core, resulting in successful sampling of loose or difficult to sample sediments.

Sediment/water slurry samples were field sieved through a 4-mm screen, briefly mixed, transferred to new glass jars with Teflon®-lined lids, and immediately placed on ice. This procedure resulted in sediment/water slurries with approximately 40 to 70% water content. Samples were shipped by overnight air to the lab, and stored in the dark at about 4° C. until used. Because of concerns about possible changes in pore water PAH concentrations during storage, GC/MS and SPME-LIF analyses were typically performed within one week of each other, and all sediments were analyzed in less than 28 days after collection. Total organic carbon (TOC) and black carbon (BC) were determined by elemental analysis (C,H,N) after acidification with HCl to remove inorganic carbonates. Samples for BC were prepared by oxidation under air at 375° C. for 24 hours in a gas chromatographic oven. (Gustafsson, O.; Haghseta, F.; Chan, C.; MacFarlane, J.; Gschwend, P. M. Quantification of the dilute sedimentary soot phase: Implications for PAH speciation and bioavailability. Environ. Sci. Technol. 1997, 31, 203-209,) incorporated herein by reference.

Sediment and pore water PAH-34 concentrations were determined in quadruplicate using GC/MS. Sediment extracts were prepared using 18-hr Soxhlet extractions. Pore water samples were prepared using centrifugation followed by flocculation, and concentrations were determined using commercially-available SPME fibers (7 μm PDMS coating, available from Supelco, Bellefonte, Pa.) specifically designed for thermal desorption into a gas chromatograph's injection port.

Both methods used 2- to 6-ring perdeuterated PAHs as analytical internal standards. Pore water TUs were calculated using octanol water coefficients (K_{OW}) as specified by the U.S. EPA, (U.S. Environmental Protection Agency. Procedures for the derivation of ESBs for the protection of benthic organisms: PAH mixtures; EPA/600/R-02/013; Office of Research and Development: Washington, D.C., 2003), incorporated herein by reference.

### 3. SMPE-LIF Determinations

The SPME sorbent used for the in situ studies was prepared by stripping the nylon buffer from a plastic (PDMS) clad fused silica optical fiber available from Fiberguide Industries, Inc (Stirling, N.J.), with hot propylene glycol for approximately 2 minutes. The remaining PDMS cladding (50 μm film thickness, 600 μm outer diameter) was found to have the lowest LIF response of any of the various PDMS materials tested. Numerous other tubings, tapes, sheets, and two-part PDMS materials from a wide range of other applications and manufacturers, were examined for autofluorescence and all had significantly higher autofluorescence than the optical fiber. (Note: It is important not to confuse the SPME fibers used for GC/MS analysis of the flocculated pore water samples described above, in the section entitled, “Sediment Collection and Characterization for GC/MS Testing,” and the SPME rods made from optical fibers used for direct insertion into the sediments followed by LIF determinations.) Each rod was cut into 2-cm lengths, rinsed with water, and stored in reverse osmosis purified water (previously determined to be clean of fluorescence via direct measurement with LIF).

SPME sorptions of the PAHs in sediment/water slurries were performed directly in the 250-mL jars used to ship the samples to the laboratory from the field. The rod was simply inserted into the center of the sediment/water slurry and the samples were kept in the dark during the exposure times. No steps were taken to prepare the field samples prior to inserting the cleaned SPME rod. In an effort to best mimic use of the rods in the field (e.g., inserting the rod into the top 10 cm of sediment, or the biologically active zone), no mixing was used. After the selected exposure time, the rod was removed from the sediment, particles were removed with a brief spray of clean water, and the PAH content was analyzed by LIF.

LIF was performed using an ultra-violet optical screening tool, UVOST® fluorometer available from Dakota Technologies, Inc (Fargo, N. Dak.). The sorbent rod was placed in a holder at 90 degrees to collinear excitation and emission fiber optics, located approximately 5 mm from the surface of the rod. This orientation provided an optical interrogation zone approximately 3 mm long at the center of the rod's length, which allowed the sorbent rod to be handled at the ends without disturbing or contaminating the section of the rod interrogated by LIF. Excitation was achieved with 5 nanosecond full width at half maximum (FWHM) pulses from a XeCl excimer laser. Since one of the advantages of this method is the ability to monitor the total alkyl and parent PAHs, excitation was performed at 308 nm in order to excite 2- to 6-ring PAHs, and to avoid fluorescence from monocyclic aromatics such as benzene and toluene. Emission wavelengths were monitored at center wavelengths of 350, 400, 450, and 500 nm (±40nm bandpass) in order to monitor emission from 2- to 6-ring PAHs, as has previously been demonstrated in soil and sediment samples, (Grundl, T. J.; Aldstadt, III, J. H.; Harb, J. G.; St. Germain, R. W.; Schweitzer, R. C. Demonstration of a method for the direct determination of polycyclic aromatic hydrocarbons in submerged sediments. Environ. Sci. Technol. 2003, 37, 1189-1197), incorporated herein by reference. Normalization of the data was based on the fluorescence response of a reference emitter (RE) prepared from a standard solution of 2- to 6-ring PAHs diluted in acetone.

### 4. Data Analysis Technique

Data from all four testing sites were evaluated using Minitab 14 statistical software available from Minitab, Inc. (State College, Pa.). The GC/MS pore water concentrations and SPME-LIF intensities were evaluated for normality using the Ryan-Joiner test for data in original and log-transformed units. Data determined to be neither normal nor log-normally distributed were transformed using ranks. Correlations between measurements were determined using either the Pearson product moment (normal data) or Spearman rank correlation (non-normal data). Principal component analysis (PCA) was used to identify which variables explained the largest percentage of the variance in the GC/MS pore water concentrations of 2- through 6-ring PAHs and LIF emission intensities at 350, 400, 450, and 500 nm. PCA was calculated from the correlation matrix.

### 5. Sediment Characteristics and PAH Concentrations

General characteristics of the manufactured gas plant (MGP) site sediments used in this study are given in Table 1 below.

TABLE 1
Summary of Sediment and Pore Water Characteristics

| | Minimum | Maximum | Median |
|---|---|---|---|
| Bulk Sediment^{a} | | | |
| total PAH₃₄ (μg/g) | | | |
| Sites A, B, C (n = 22) | 9 | 768 | 166 |
| Site D (n = 11) | 57 | 902 | 135 |
| Site E (n = 10) | 46 | 1057 | 184 |
| 2- and 3-ring PAHs/total PAH₃₄, %^{b} | | | |
| Sites A, B, C | 37 | 65 | 51 |
| Site D | 68 | 96 | 90 |
| Site E | 8 | 19 | 13 |
| Total Organic Carbon (TOC)^{c} | 0.14 | 5.3 | 1.2 |
| Black Carbon (BC)^{c} | 0.06 | 2.1 | 0.47 |
| Fraction (BC/TOC)^{c} | 0.11 | 0.88 | 0.37 |
| Sediment Pore Water | | | |
| total PAH₃₄ ng/L | | | |
| Sites A, B, C | 2 | 501 | 16 |
| Site D | 56 | 1429 | 597 |
| Site E | 1 | 27 | 2 |

^{a}Sediment PAH concentrations are on a dry weight basis.
^{b}The sum concentration of all 2- and 3-ring PAHs divided by the total PAH₃₄ concentration.
^{c}All sites.

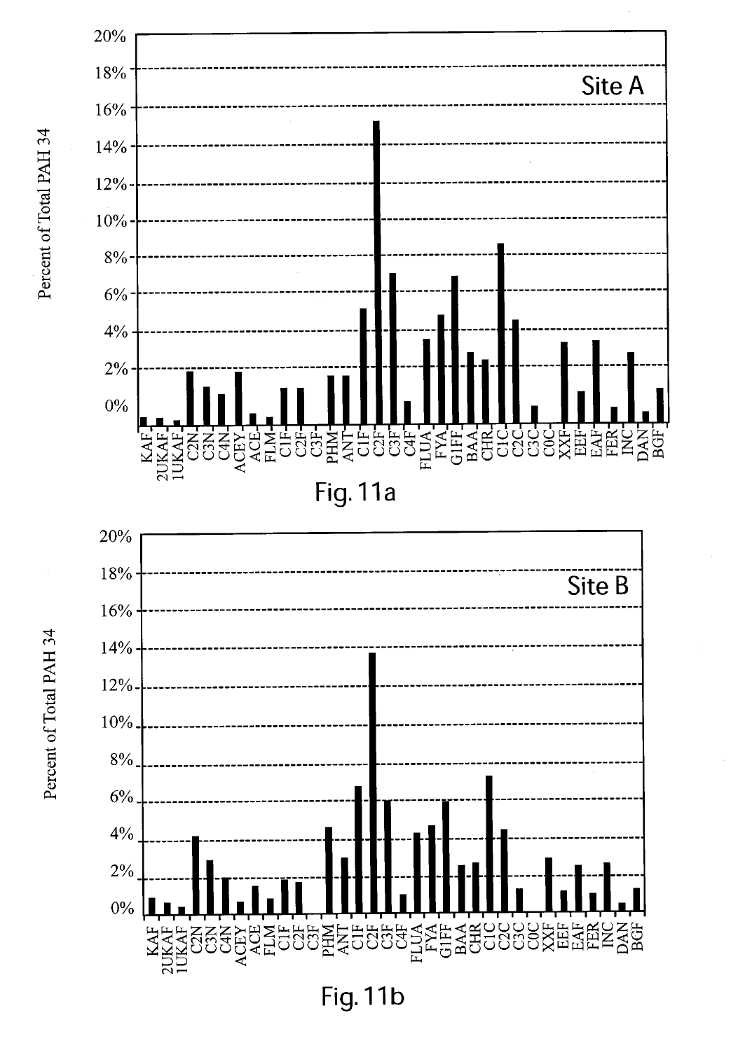

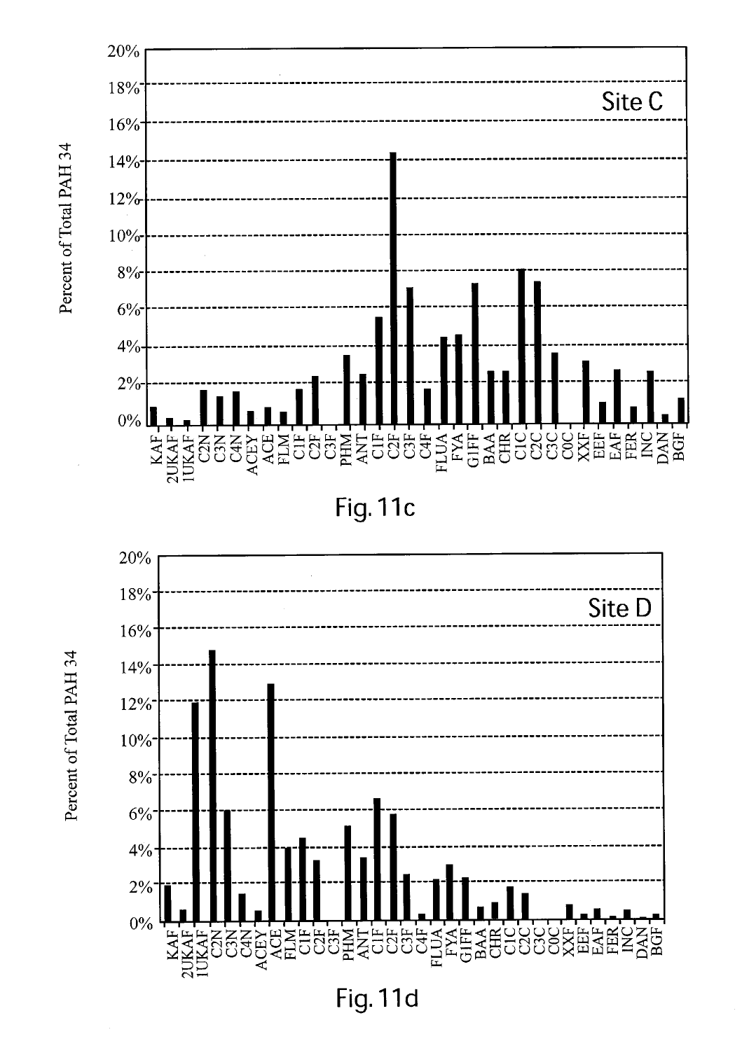

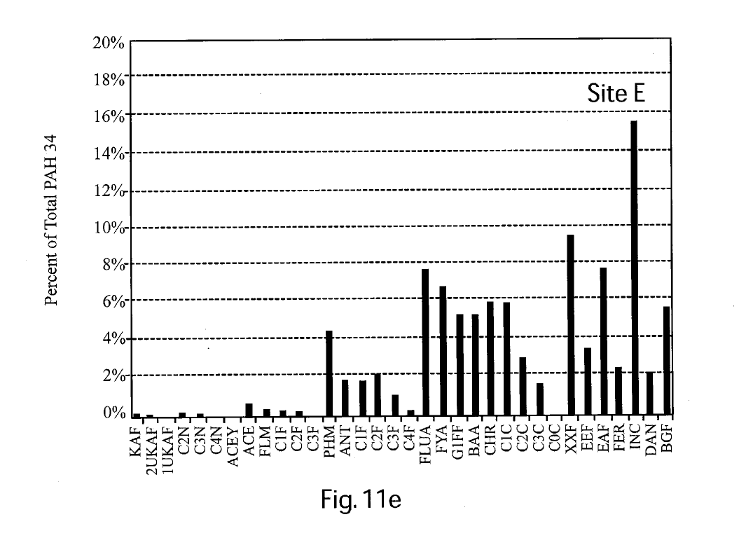

Relative distributions of each of the PAH-34 parent and alkyl groups are shown in FIG. 11. Sites A, B, and C involved MGP surface sediments, and show PAH ring-size distributions that are typical of the vast majority of the 230 sediments that were analyzed for sediment and pore water PAH-34, (Hawthorne, S. B.; Grabanski, C. B.; Miller, D. J. Measured partitioning coefficients for parent and alkyl polycyclic aromatic hydrocarbons in 114 historically contaminated sediments: Part 1. K_{OC} values. Environ. Toxicol. Chem. 2006, 25, 2901-2911. Hawthorne, S. B.; Azzolina, N. A.; Neuhauser, E. F.; Kreitinger, J. P. Predicting bioavailability of sediment polycyclic aromatic hydrocarbons to Hyalella azteca using equilibrium partitioning, supercritical fluid extraction, and pore water concentrations. Environ. Sci. Technol. 2007, 41, 6297-6304), both incorporated herein by reference.

Site D was also from an MGP location, but samples consisted of subsurface cores collected from depths greater than one foot below the sediment surface. Site D was included in this study because it showed the highest relative concentrations of low molecular weight PAHs of any of the 14 MGP and aluminum smelter sites analyzed to date. Lastly, Site E included surface sediments from an aluminum smelting site that historically used coal tar pitch in its manufacturing processes. Site E was selected because it represented the highest relative concentrations of high molecular weight PAHs from the sites studied to date. For the 58 sediments used in the present study, PAH-34 concentrations ranged from typical urban background concentrations of a few μg/g to impacted sediments as high as 1100 μg/g PAH-34. Total organic carbon (TOC) ranged from 0.14 to 5.3 wt %, and BC ranged from 0.06 to 2.1% (See Table 1, above). Sediment textures ranged from coarse sand to fine silt and clay.

Twenty of the 58 sediments had non-aqueous phase liquid (NAPL) observed in the field during sample collection, and confirmed in the laboratory. However, no attempt was made to remove NAPL droplets prior to SPME-LIF analysis, since no such alteration of the sediments would be possible in an in situ field approach.

### 6. Effect of Exposure Time on LIF Response

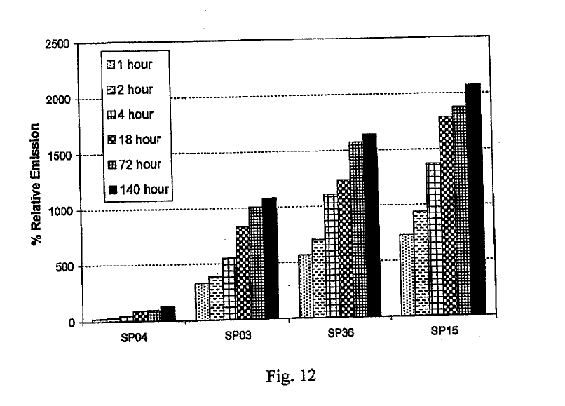

The effect of the SPME exposure time to the sediment/water slurry samples on the SPME-LIF response is shown in FIG. 12. Even under the static (no mixing) conditions used to mimic in situ sampling, sorption occurs fairly rapidly. For example, after only one hour the SPME-LIF signals were about 30% of the values attained after 140 hours of exposure, and after 18 hours the response averaged 77±7% of the values attained after 140 hours. Since 18 hours represents a reasonable time frame for deploying and retrieving multiple in situ SPME devices in the field, the 18 hour exposure time was chosen for subsequent studies unless otherwise noted. It should also be noted that useful survey data can be achieved with quite short exposure times. For example, with the eight sediments used in the time studies (including those in FIG. 12), the linear correlation between the SPME-LIF responses obtained after one hour compared to the responses at either 18 or 140 hours was very strong (r²=0.96). These results indicate that useful site mapping survey data could be obtained during field studies on hour time frames, which would allow near real-time adaptive management of field sampling and analysis plans.

### 7. Effect of Sediment Volume on LIF Response

In order for the SPME approach to apply in the field, the concentrations of PAHs sorbed into the rod coating should be relatively independent of sample size; i.e., a rod placed in the sediment of a large body of water such as a lake or river should have the same PAH concentrations and the same fluorescence response as a rod placed in a small jar with a few (about 3 to about 7 milliliters) of the same sediment. The sample size should be of sufficient size in which to immerse a coated portion of the sampler rod. For example, the immediate surroundings just around the SPME will contain enough PAHs to reach equilibrium or detectable amounts. In essence, this is the same as saying the SPME extraction must be non-depletive to the exposed sediment/pore water slurry PAH concentrations, as is required for other equilibrium based in situ methods, (Cornelissen, G.; Pettersen, Ar.; Broman, D.; Mayer, P.; Breedveld, G. D. Field testing of equilibrium passive samplers to determine freely dissolved native polycyclic aromatic hydrocarbon concentrations. Environ. Toxicol. Chem. 2008, 27(3) 499-508. Hunter, W.; Xu, Y.; Spurlock, F.; Gan, J. Using disposable polydimethylsiloxane fibers to assess the bioavailability of permethrin in sediment. Environ. Toxicol. Chem. 2008, 27(3), 568-575. Jonker, M. T. O.; Van Der Heijden, S. A.; Kreitinger, S., Hawthorne, S. B. Predicting PAH bioaccumulation and toxicity in earthworms exposed to manufactured gas plant soils with solid-phase microextraction. Environ. Sci. Technol. 2007, 41, 7472-7478. Styrishave, B.; Mortensen, M.; Krogh, P. H.; Andersen, O.; Jensen, J. Solid-phase microextraction (SPME) as a tool to predict the bioavailability and toxicity of pyrene to the springtail, Folsomia candida, under various soil conditions. Environ. Sci. Technol. 2008, 42, 1332-1336), all incorporated herein by reference.

“Non-depletive” extraction methods such as those used in cited references above refer to the use of sorbents in mixed sediment/water conditions in ratios such that the concentration of each target analyte in water at equilibrium does not significantly change whether the sorbent is present or not. This is normally defined in most literature as the sorbent removing less than 5% of the number of target molecules in sediment/water slurry. For hydrophobic organics like PAHs, the vast majority (about 99.9% to 99.9999%) of individual molecules reside primarily on the sediment, with much smaller numbers of molecules in the associated pore water. Thus, when a “non-depletive” sorbent is added, the dissolved PAHs that are collected from the water are replaced by sediment/water partitioning. Under the non-depletive conditions, the 3-way equilibrium among sediment/pore water/sorbent results in essentially the same pore water concentrations, but much higher concentrations in the sorbent than if the sorbent was simply in contact with the water. This allows for the sensitivity of the methods cited in the references above, since the sediment acts as a supplier to re-establish the original pore water concentrations (as long as the sorbent does not remove more than 5% of the total mass of any particular PAH).

A comparison of the LIF signal from pore water isolated from 10 of the sediments was much lower. To emphasize, the sensitivity of non-depletive sorbent approaches and the ability to determine dissolved PAH concentrations requires the three-way equilibrium among sediment/pore water/sorbent under non-depletive conditions. This does not occur for isolated pore water.

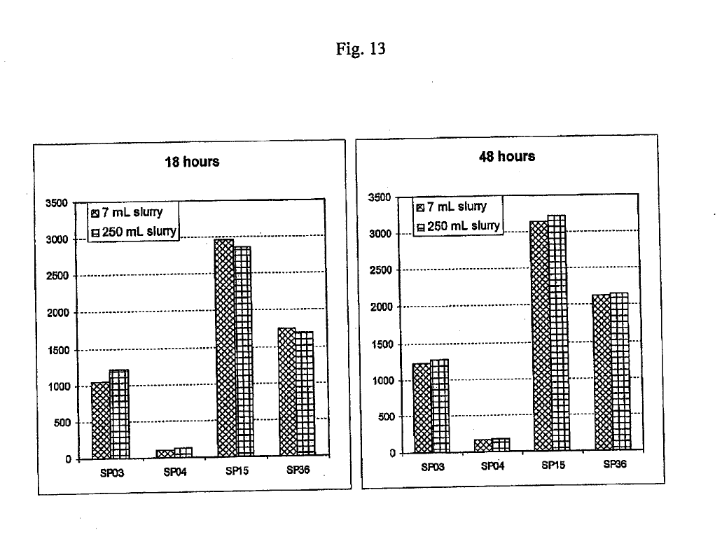

To test if this sample size independence (i.e., non-depletive) requirement was met, the four sediments that were used for the time study in FIG. 12 were exposed to 7 mL and 250 mL sediment/water slurry samples for 18 and 48 hours. See Mayer, P.; Tolls, J.; Hermens, J; Mackay, D. Equilibrium Sampling Devices. Environ. Sci. Technol. 2003, 37, 185A-191A, incorporated herein by reference. After 18 hours the fluorescence signal in the 7 mL samples averaged 96±11% of the signal in the 250 mL samples, and after 48 hours the signals from the 7 mL samples averaged 98±6% of those for the 250 mL samples FIG. 13 shows the effect of sample volume on SPME-LIP response after 18 and 48 hours.

These results demonstrate that there is no dependence on sample size that can be measured compared to the method reproducibility (which has a relative standard deviation (RSD) of about 8% based on the LIF response of five rods placed in the same sediment sample for 18 hours). Therefore, a rod exposed to sediment in the field will accurately reflect the pore water PAHs in a small sample taken from the same location, and vice-versa.

### 8. Background and Detection Limit

Advantageously, this method attains a detection limit for PAHs corresponding to one

TU (or lower) as defined by the EPA narcosis model, (See U.S. Environmental Protection Agency. Procedures for the derivation of ESBs for the protection of benthic organisms: PAH mixtures; EPA/600/R-02/013; Office of Research and Development: Washington, D.C., 2003), a value which corresponds to a total PAH-34 water concentration of about 10 ng/mL for a sediment that has a typical distribution of PAHs from an MGP site. With the LIF system, the fluorescence response does not limit sensitivity; rather the major limitation to achieving low detection limits is the background fluorescence from the PDMS sorbent material. The material chosen for this study was the PDMS found to have the lowest background of those tested. Since alkyl two- and three-ring PAHs contribute the highest pore water concentrations and generally account for the most TUs of the PAH-34 list, (Hawthorne, S. B.; Azzolina, N. A.; Neuhauser, E. F.; Kreitinger, J. P. Predicting bioavailability of sediment polycyclic aromatic hydrocarbons to Hyalella azteca using equilibrium partitioning, supercritical fluid extraction, and pore water concentrations. Environ. Sci. Technol. 2007, 41, 6297-6304), it would be desirable to prepare solutions containing “standard” alkylated isomeric clusters for calibration and determining detection limits. However, no standards of the alkylated isomeric clusters currently exist, and their production from pure compounds is not currently possible because of the several hundreds of isomers present in PAH contaminated materials from both petrogenic and pyrogenic sources. (Hawthorne, S. B.; Miller, D. J.; Kreitinger, J. P. Measurement of ‘total’ PAH concentrations and toxic units used for estimating risk to benthic invertebrates at manufactured gas plant sites. Environ. Toxicol. Chem. 2006, 25, 287-296.)

Therefore, the SPME-LIF method detection limit was estimated by comparing SPME-LIF response to the concentrations measured by the pore water PAH-34 GC/MS method, (Hawthorne, S. B.; Grabanski, C. B.; Miller, D. J.; Kreitinger, J. P. Solid-phase microextraction measurement of parent and alkyl polycyclic aromatic hydrocarbons in milliliter sediment pore water samples and determination of K_{DOC} values. Environ. Sci. Technol. 2005, 39, 2795-2803), on several sediment samples that had low pore water concentrations. With the preparation described above, the PDMS on the rod selected for this study showed background signals at about 10% relative emission (on the scale shown in FIG. 12). Based on a 3:1 signal to noise ratio, the SPME-LIF method currently has a detection limit for total PAH-34 in pore water of about 2 ng/mL, which corresponds to about 0.2 TUs. Since these values are below typical urban background levels in sediments (Hawthorne, S. B.; Azzolina, N. A.; Neuhauser, E. F.; Kreitinger, J. P. Predicting bioavailability of sediment polycyclic aromatic hydrocarbons to Hyalella azteca using equilibrium partitioning, supercritical fluid extraction, and pore water concentrations. Environ. Sci. Technol. 2007, 41, 6297-6304), the method is sufficiently sensitive to use at industrial and urban sites. However, obtaining PDMS material that has a lower background signal would further reduce the method detection limit, since the LIF signal is still reasonably intense at this background level.

Fluorescence from dissolved organic matter (DOM) has been a major obstacle to direct fluorescence determinations of PAHs in water (Kuo, D. T. F.; Adams, R. G.; Rudnick, S. M.; Chen, R. F.; Gschwend, P. M. Investigating desorption of native pyrene from sediment on minute- to month-timescales by time-gated fluorescence spectroscopy. Environ. Sci. Technol. 2007, 41(22), 7752-7758. Nahorniak, M. L.; Booksh, K. S. Excitation-emission matrix fluorescence spectroscopy in conjunction with multiway analysis for PAH detection in complex matrices. Analyst, 2006, 131, 1308-1315. Valero-Navarro, A.; Fernández-Sánchez, Medina-Castillo, A. L.; Fernändez-Ibáñez, F.; Segura-Carretero, A.; Ibáñz, J. M.; Fernández-Gutiérrez. A rapid, sensitive screening test for polycyclic aromatic hydrocarbons applied to Antarctic water. Chemosphere 2007, 67, 903-910. Rudnik, S. M.; Chen, R. F. Laser-induced fluorescence of pyrene and other polycyclic aromatic hydrocarbons (PAH) in seawater. Talanta 1998, 47, 907-919. Kotzick, R.; Niessner, R. Application of time-resolved, laser-induced and fiber-optically guided fluorescence for monitoring of a PAH-contaminated remediation site. Fresenius J. Anal. Chem. 1996, 354, 72-76), all incorporated herein by reference, but does not appear to affect the SPME-LIF approach since DOM is too polar to preferentially sorb into (or onto) the non-polar PDMS.

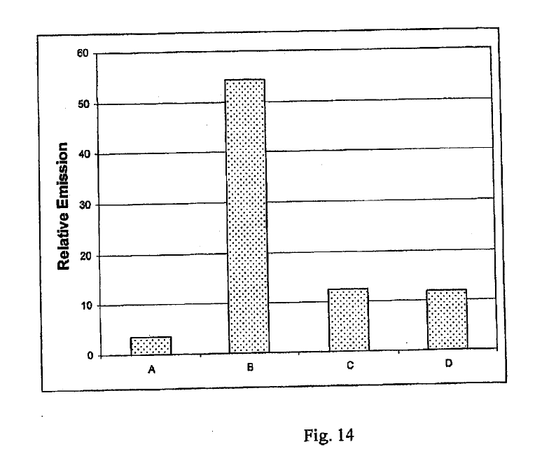

For example, a rod soaked for 18 hours in a solution of 9 mg/mL Suwannee River fulvic acid in water showed no detectable change in LIF response from a duplicate rod soaked in clean water, even though the fulvic acid water solution showed an LIF response several times the rod background response. FIG. 14 provides a chart illustrating the direct LIF response for pure water (A) and water with 9 mg/L of fulvic acid (B) compared to the SPME-LIF response (18 hour) for pure water (C) and 9 mg/L fulvic acid (D). Similarly, water samples equilibrated for 24 hours (1:3 wt. to wt. ratio in water) with manure, peat moss, and a 13 wt. % TOC agricultural soil showed no increase in SPME-LIF response compared to clean water, further demonstrating that the SPME sorbent efficiently excludes background fluorescence from natural organic matter.

Potential effects of DOM were also investigated by measuring the SPME-LIF response of 15 clean background sediments that were collected in unimpacted areas from the same five sites (in addition to the 43 sediments used in the remainder of this study) that had PAH-34 pore water concentrations (as measured by the GC/MS method) below the SPME-LIF detection limit discussed above. After the 18 hour exposure of the SPME rod to the sediment/slurry mix, the only cleaning step was a brief rinse with clean water. For all of these samples, no significant fluorescence above the rod background was observed relative to variation seen in repeat measurements. The lack of SPME-LIF response in these uncontaminated sediments also demonstrates that any colloids which may stick to the rod surface after rinsing do not cause a detectable change in the LIF signal.

### 9. SPME-LIF Response Compared to Laboratory PAH-34 GC/MS Analyses

A comparison of the 18 hr SPME-LIF signals with the total sediment and total pore water PAH-34 concentrations, and with the total PAH toxic units calculated from the EPA's narcosis model (U.S. Environmental Protection Agency. Procedures for the derivation of ESBs for the protection of benthic organisms: PAH mixtures; EPA/600/R-02/013; Office of Research and Development: Washington, D.C., 2003) was initially performed on a site-by-site basis. These plots showed general agreement for sites A, B, C, and E, but significant deviations for site D (as discussed below). Therefore, subsequent data analysis was performed with the combined data from sites A, B, C, and E, but with the data from site D handled separately unless otherwise noted. It should also be noted that the EPA's hydrocarbon narcosis model predicts mortality to Hyalella azteca when PAH-34 water concentrations are high enough to contribute one toxic unit (equivalent to 2.2 μmole/g lipid), so the important range for accuracy of the SPME-LIF method might initially be considered about <1 to 3 toxic units. However, a recent study of 97 PAH-impacted field sediments demonstrated that no mortality occurs below 5 toxic units, and that the important range for distinguishing toxic versus non-toxic samples is from about 5 to 30 toxic units (Hawthorne, S. B.; Azzolina, N. A.; Neuhauser, E. F.; Kreitinger, J. P. Predicting bioavailability of sediment polycyclic aromatic hydrocarbons to Hyalella azteca using equilibrium partitioning, supercritical fluid extraction, and pore water concentrations. Environ. Sci. Technol. 2007, 41, 6297-6304). A similar result was obtained by a separate study using pure fluoranthene under controlled laboratory conditions (Schuler, L. J.; Landrum, P. F.; Lydy, M. Comparative toxicity of fluoranthene and pentachlorobenzene to three freshwater invertebrates. Environ. Toxicol. Chem. 2006, 25, 985-994), incorporated herein by reference. Therefore, evaluation of SPME-LIF in subsequent discussions focuses on PAH-34 concentrations contributing about 5 to 30 toxic units.

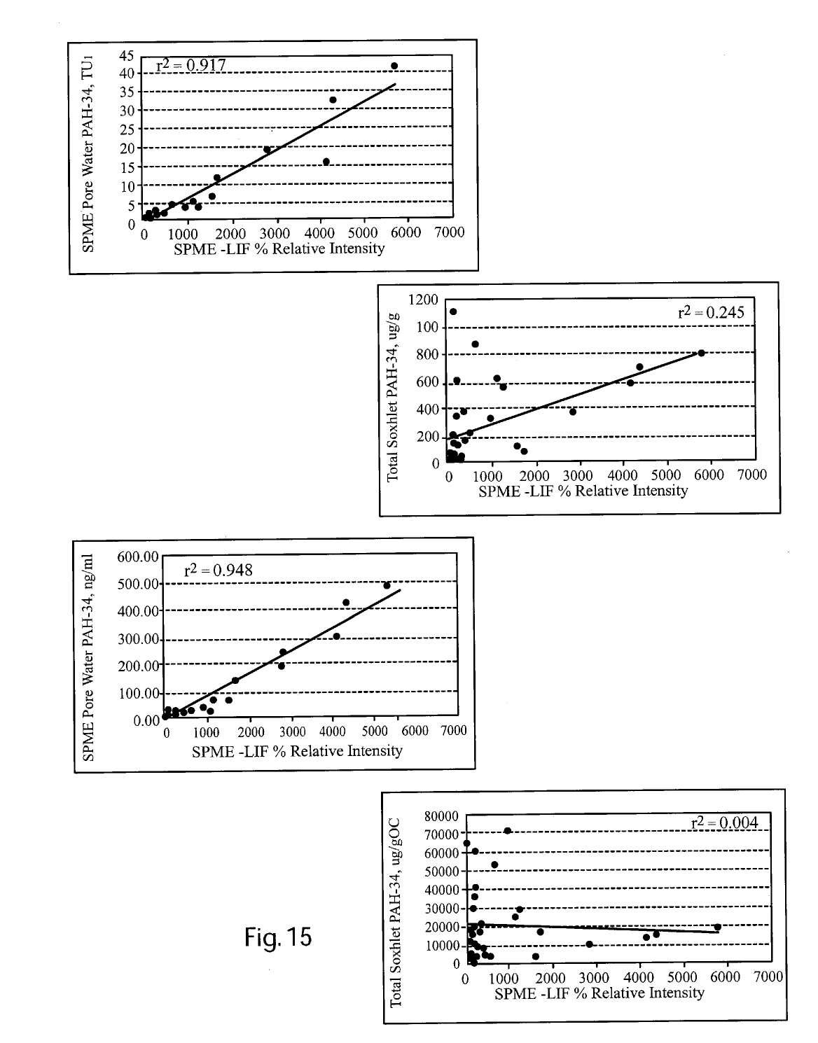

FIG. 15 shows the linear correlations between the SPME-LIF response and the pore water TUs, total dissolved pore water concentrations, and the total sediment concentrations for the PAH-34 from sites A, B, C, and E (33 surface sediments). For both total pore water TUs and PAH-34 concentrations, the Pearson correlation is quite good (r²=0.92 and 0.95, respectively). However, the correlation between the total sediment concentrations and the SPME-LIF signal is low (r²=0.245), as is the correlation with sediment concentrations expressed on an organic carbon (OC) basis (r²=0.004). This poor correlation with the sediment concentrations is expected, since the sorbent coating approaches equilibrium with the pore water fraction, and it is known that pore water PAH concentrations can not be accurately estimated using literature K_{OC} values and sediment PAH concentrations for MGP and other historically-contaminated sediments (Jonker, M. T. O.; Koelmans, A. A. Sorption of polycyclic aromatic hydrocarbons and polychlorinated biphenyls to soot and soot-like materials in the aqueous environment: Mechanistic considerations. Environ. Sci. Technol. 2002, 36, 3725-3734. Cornelissen, G.; Gustafsson, O.; Bucheli, T. D.; Jonker, M. T. O.; Koelmans, A. A.; van Noort, P. C. M. Extensive sorption of organic compounds to black carbon, coal, and kerogen in sediments and soils: mechanisms and consequences for distribution, bioaccumulation, and biodegradation. Environ. Sci. Technol. 2005, 39, 6881-6895. Khalil, M. F.; Ghosh, U.; Kreitinger, J. P. Role of weathered coal tar pitch in the partitioning of polycyclic aromatic hydrocarbons in manufactured gas plant site sediments. Environ. Sci. Technol. 2006, 40, 5681-5687. Hawthorne, S. B.; Grabanski, C. B.; Miller, D. J. Measured partitioning coefficients for parent and alkyl polycyclic aromatic hydrocarbons in 114 historically contaminated sediments: Part 1. K_{OC} values. Environ. Toxicol. Chem. 2006, 25, 2901-2911. Lohmann, R.; MacFarlane, J. K.; Gschwend, P. M. Importance of black carbon to sorption of native PAHs, PCBs, and PCDDs in Boston and New York harbor sediments. Environ. Sci. Technol. 2005, 39, 141-148), all incorporated herein by reference.

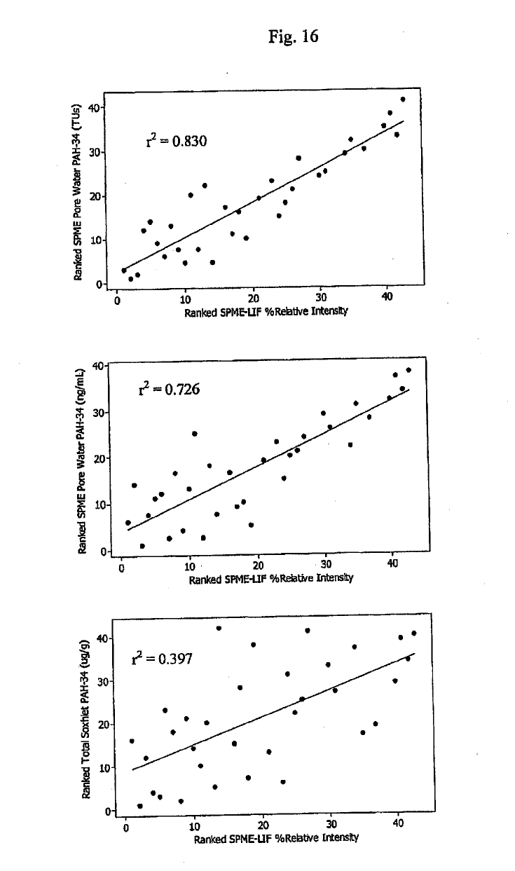

Since several of the 33 sediments shown in FIG. 15 had PAH-34 concentrations and SPME-LIF intensities that were neither normal nor log-normally distributed, a Spearman rank correlation was also done, and yielded similar results. For the pore water TUs, PAH-34 concentrations, and sediment concentrations, the Spearman rank correlation coefficients were 0.83, 0.73, and 0.40, respectively. FIG. 16 provides graphs of Spearman rank correlations for SPME-LIF responses compared to total pore water toxic units (top), total pore water PAH-34 concentrations (middle), and total sediment PAH-34 concentrations (Sites A, B, C, and E).

### 10. Effect of PAH Molecular Weight Distribution

As noted above, sediments from sites A, B, and C have PAH molecular weight distributions that are typical of the vast majority of 230 sediments we have analyzed from 16 MGP and related sites. However, different PAH distributions are likely to be encountered from some locations, and it is important to understand the effect of PAH distribution on the SPME-LIF response. As shown in Table 1 and FIG. 11, the sediments from site D had a much higher proportion of low molecular weight PAHs than is typical for surface sediments from MGP sites, as might be expected since the sediments from site D were obtained from cores collected below the sediment surface and had therefore been subjected to less weathering than the surface sediments from sites A, B, and C. Thus, while naphthalene and alkyl naphthalenes normally account for about 10% of the total PAH-34 sediment concentrations, they account for about 40% for site D sediments. In contrast, sediments at Site E consisted of higher molecular weight PAHs, and only about 2% of the sediment PAHs consist of naphthalene and alkyl naphthalenes, as might be expected since the major source of PAHs at Site E were from coal tar pitch, which consists of higher molecular weight PAHs than typical MGP tars.

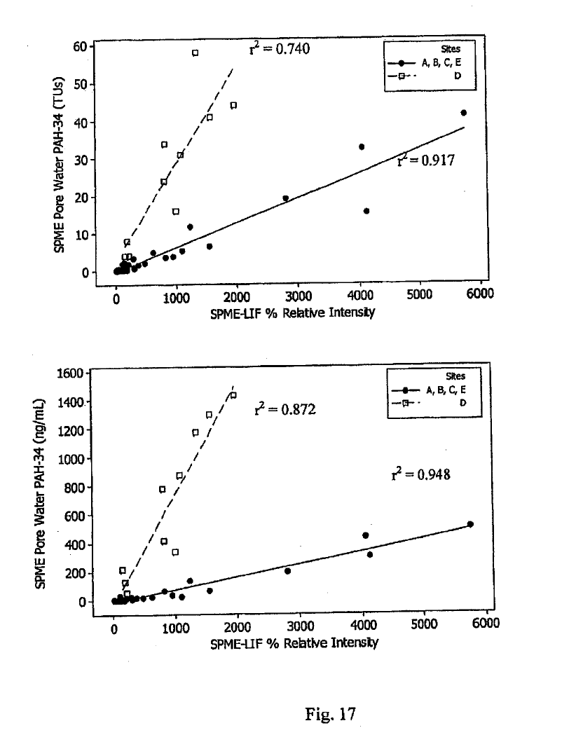

However, as shown in FIG. 15, the pore water PAH data from Site E do correlate with those from the sites A, B, and C despite the differences in molecular weight distribution. For the subsurface core samples from site D, even though the correlation of SPME-LIF response with total pore water TUs and total pore water PAH-34 remains quite good (r²=0.74 and 0.87, respectively), the SPME-LIF response is significantly lower for the site D samples, as evidenced by the slopes of the least squares regression lines. For site D, the slope of the total pore water PAH-34 concentrations versus LIF response (See FIG. 17) is nine-fold steeper than for sites A, B, C, and E. Similarly, the slope of the total pore water TUs is four-fold higher for site D. That is, to get the same SPME-LIF signal for site D as for the other more typical sites shown in FIG. 15, the pore water must have nine times the total dissolved PAH concentrations, or four times higher TUs.

These results demonstrate that, as might be expected, some knowledge about the molecular weight distribution of PAHs at a particular site will be needed to verify any quantitative determinations of pore water PAH-34 concentrations or toxic units based on SPME-LIF response at different sites.

### 11. Effect of Monitoring Wavelength

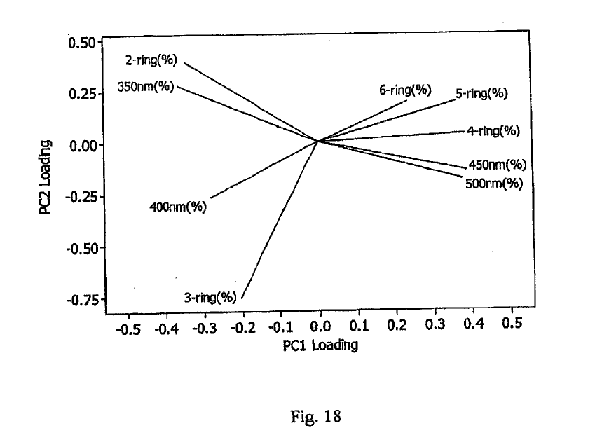

As described above, the LIF emission wavelengths were chosen at 350, 400, 450, and 500 nm to monitor all 2- to 6-ring PAHs with similar sensitivities (Kotzick, R.; Niessner, R. Application of time-resolved, laser-induced and fiber-optically guided fluorescence for monitoring of a PAH-contaminated remediation site. Fresenius J. Anal. Chem. 1996, 354, 72-76. Owen, C. J.; Axler, R. P.; Nordman, D. R.; Schubauer-Berigan, M.; Lodge, K. B.; Shubauer-Berigan, J. P. Screening for PAHs by fluorescence spectroscopy: a comparison of calibrations. Chemosphere 1995, 31, 3345-3356), both incorporated herein by reference. Based on standard fluorescence spectra of standard pure PAHs, the emission wavelength at 350 nm primarily focuses on 2-ring PAHs, while the higher wavelengths monitor increasing higher molecular weight PAHs. However, real-world MGP samples have hundreds to thousands of individual parents and alkyl isomers (Hawthorne, S. B.; Miller, D. J.; Kreitinger, J. P. Measurement of ‘total’ PAH concentrations and toxic units used for estimating risk to benthic invertebrates at manufactured gas plant sites. Environ. Toxicol. Chem. 2006, 25, 287-296.), as well as heteroatom-containing aromatics including (but not limited to) 2- to 4-ring furans, thiophenes, and pyroles. Therefore, principal component analysis (PCA) was used to evaluate the emission wavelength relationship to the relative percentage of 2- to 6-ring PAHs for the complex mixture of alkyl and parent PAHs found at these sites. The first two principal components (PCs) accounted for 81% of the total variance in emission wavelength and PAH ring size. A loading plot of the first 2 PCs shows that 2- and 3-ring PAHs are tightly associated with 350 and 400 nm emissions, while the higher molecular weight PAHs are associated with the longer emission wavelengths, which verifies the expectations based on pure compound emission spectra (FIG. 18).

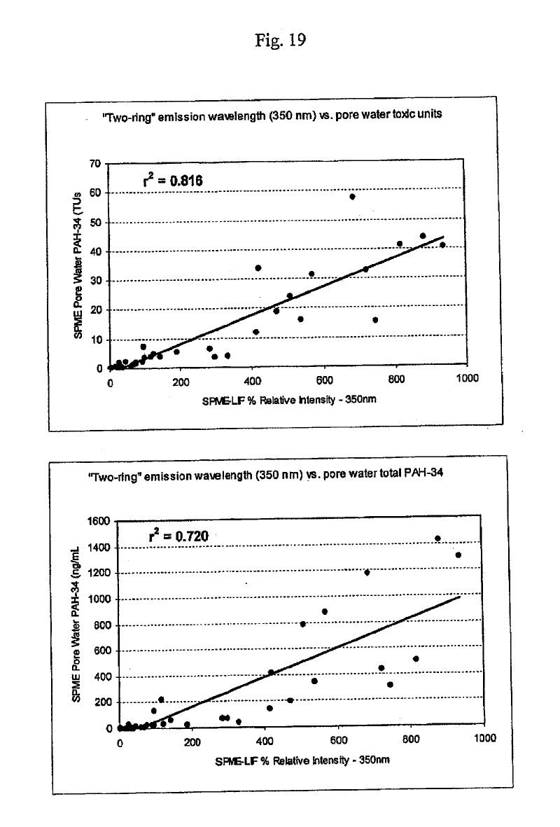

Since (as discussed above), the 2- and 3-ring PAHs dominate pore water PAH concentrations and related toxic units, we investigated the use of only 350 or 350 and 400 nm emission signals. Interestingly, the differences in SPME-LIF response previously shown by site D are reduced compared to the other four sites when only the 350 nm emission is monitored as shown in FIG. 19 (the plot from the sum of 350 and 400 nm looks similar). Linear correlation coefficients (r²) for the five combined sites are 0.72 for the total pore water PAH-34 concentration, but increase to 0.81 for the pore water TUs. Similarly, the Spearman rank correlations are 0.81 for the pore water PAH-34 concentrations, and 0.88 for the pore water TUs.

The 350 nm emission coupled with 308 nm excitation data clearly suggests that SPME-LIF should be useful for screening MGP and related sites for pore water PAHs, without the need for prior knowledge of the PAH distribution. Substantial savings of analytical costs would be achieved if high numbers of samples were pre-screened with SPME-LIF to determine those that are certainly “clean” or obviously toxic, and submitting those that fall within the possibly toxic range for confirmatory analysis. The results of combining the four emission wavelength data (as in FIG. 12) demonstrate that reasonable quantitative data can be obtained with the technique, as long as the PAH distribution at the site is not highly unusual for an MGP site.

The results also demonstrate that the SPME-LIF approach can be used to obtain semi-quantitative results with exposure times as short as one hour, which could greatly aid field real-time adaptive management of sample location selections and analytical programs. Since nearly one-half (20 of 43) of the impacted sediments had NAPL phases present (including samples with high and low PAH-34 concentrations), the good correlation with dissolved pore water concentrations shows that the SPME-LIF procedure is not degraded by the presence of a NAPL phase, as is desirable for any field applications of the method.

The SPME-LIF approach can be used on-site to rapidly map the relative PAH pore water concentrations, and those results could be used to select sampling areas for more complete testing such as pore water PAH-34 by GC/MS and biological toxicity studies. The coated rods are inexpensive and the LIF measurement requires only a few min per sample using instrumentation similar to that already routinely deployed in field studies. In addition, no solvents or other hazardous materials are needed to perform SPME-LIF in the field.

From the above description and drawings, it will be understood by those of ordinary skill in the art that the particular embodiments shown and described are for purposes of illustration only and are not intended to limit the scope of the present invention. Those of ordinary skill in the art will recognize that the present invention may be embodied in other specific forms without departing from its spirit or essential characteristics. References to details of particular embodiments are not intended to limit the scope of the invention.

What is claimed is:

1. A method for analyzing aromatic hydrocarbons dissolved in water comprising:
providing a substrate coated with a thin film layer of a material, wherein the material has a high affinity for at least one aromatic hydrocarbon, the material is substantially optically transparent, and the material has near-zero auto fluorescence;
inserting the coated substrate directly into an environmental location comprising water;
waiting for an exposure time permitting at least one aromatic hydrocarbon to absorb into the thin film layer;
retrieving the coated substrate from the environmental location;
removing any non-absorbed matter from the coated substrate, and;
performing fluorescence analysis on the coated substrate to detect aromatic hydrocarbons present in the thin film layer.

2. The method of claim 1, wherein the material of the thin film layer is polydimethylsiloxane.

3. The method of claim 2, wherein the polydimethylsiloxane has a thickness of about 50 μm.

4. The method of claim 1, wherein the fluorescence analysis is laser induced fluorescence (LIF) analysis.

5. The method of claim 4, wherein the LIF analysis has a detection limit for aromatic hydrocarbons of about 0.2 ng/mL.

6. The method of claim 1, wherein the aromatic hydrocarbons are polycyclic aromatic hydrocarbons.

7. The method of claim 6, wherein the polycyclic aromatic hydrocarbons are bioavailable polycyclic aromatic hydrocarbons.

8. The method of claim 6, wherein the fluorescence analysis on the coated substrate detects 2-ring and 3-ring polycyclic aromatic hydrocarbons present in the thin film layer.

9. The method of claim 1, wherein the aromatic hydrocarbons are 2-ring to 6-ring polycyclic aromatic hydrocarbons.

10. The method of claim 1, wherein the aromatic hydrocarbons are monocyclic aromatic hydrocarbons.

11. The method of claim 1, wherein concentrations of aromatic hydrocarbons absorbing into the coating layer are relatively independent of water sample size.

12. The method of claim 1, wherein concentrations of aromatic hydrocarbons absorbing into the thin film layer occur independently of the presence of non-aqueous phase liquids.

13. The method of claim 1, wherein concentrations of aromatic hydrocarbons absorbing into the thin film layer are independent of the presence of dissolved organic matter.

14. The method of claim 1, wherein the substrate is an optical fiber.

15. The method of claim 1, wherein the substrate is magnetic.

16. A method for analyzing aromatic hydrocarbons dissolved in water comprising:
providing a substantially optically transparent substrate coated with a thin film layer of a material, wherein the material has a high affinity for at least one aromatic hydrocarbon, the material is substantially optically transparent, and the material has near-zero auto fluorescence;
inserting the coated substrate into an environmental location comprising water;
waiting for an exposure time permitting at least one aromatic hydrocarbon to absorb into the thin film layer to equilibrium;
retrieving the coated substrate from the environmental location;
removing any non-absorbed matter from the coated substrate, and;
performing analysis on the coated fiber with a fluorometer to detect aromatic hydrocarbons present in the thin film layer.

17. A method for analyzing aromatic hydrocarbons present in a fluid sample matrix, comprising:
providing a vessel;
providing a thin film layer in the vessel, wherein the thin film layer comprises polydimethylsiloxane that at least one aromatic hydrocarbon has a high affinity for and that is substantially optically transparent and has near-zero autofluorescence in the vessel;
collecting a fluid sample matrix from an environmental location;
placing the fluid sample matrix in the vessel;
periodically exposing at least one sampling point on the thin film layer to an excitation light and sensing a corresponding laser induced fluorescence (LIF) response;
storing a time sequence of LIF responses from the at least one sampling point; and
determining from the time sequence of LIF responses when an equilibrium has been sufficiently achieved for absorption of the at least one aromatic hydrocarbon into the thin film layer.

18. The method of claim 17, wherein the thin film layer is coated on an inner surface of the vessel.

19. The method of claim 18, wherein a plurality of coated vessels are provided on a carousel for collecting sample matrices over time.

20. The method of claim 17, wherein the vessel is at least one of a jar, a bag or a tube.

21. The method of claim 17, wherein the vessel is made from a substantially optically transparent material and the LIF exposing and sensing occurs with light passing through the vessel.

22. The method of claim 17, wherein the vessel has a substantially optically transparent portion, and the thin film layer is coated on the portion.

23. A method for analyzing aromatic hydrocarbons dissolved in water comprising:
providing a substrate coated with a thin film layer of a material, wherein the material has a high affinity for at least one aromatic hydrocarbon, the material is substantially optically transparent, and the material has near-zero auto fluorescence;
forming the coated substrate as a recording medium stored on a spool or in a cassette;
transporting the recording medium having a plurality of coated segments in and out of an environmental location comprising water;
exposing one or more of the plurality of coated segments to absorption of aromatic hydrocarbons from the water into the one or more coated segments;
successively drawing the plurality of coated segments into an analyzer, and;
analyzing an exposed coated segment of recording medium with a fluorometer to detect aromatic hydrocarbons present in the thin film layer.

24. The method of claim 23, wherein the recording medium is in the form of a tape, wire or string.

25. The method of claim 23, wherein the material of the thin film layer comprises polydimethylsiloxane.
